(19) United States
(12) Patent Application Publication
(10) Pub. No.: US 2006/0127339 A1
(43) Pub. Date: Jun. 15, 2006

(54) Two-coat makeup product with improved staying power, uses thereof and makeup kit comprising this product

(76) Inventors: Bruno Bavouzet, Gentilly (FR); Laure Le Chaux, L'Hay Les Roses (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/209,714

(22) Filed: Aug. 24, 2005

Related U.S. Application Data
(60) Provisional application No. 60/605,575, filed on Aug. 31, 2004.
(60) Provisional application No. 60/624,550, filed on Nov. 4, 2004.

(30) Foreign Application Priority Data
Aug. 24, 2004 (FR) 0451896
Oct. 21, 2004 (FR) 0411209

(51) Int. Cl. A61K 8/89; A61K 8/81

(52) U.S. Cl. 424/70.12; 424/70.16

(57) ABSTRACT

The present disclosure relates to a cosmetic makeup product comprising at least one first and at least one second composition, wherein the at least one first composition comprises a physiologically acceptable medium, and optionally an acrylic polymer, and the at least one second composition comprises at least one silicone polymer with a viscosity, measured according to standard ASTM D-445, ranging from 10 cSt to 10,000 cSt. The present disclosure also relates to a makeup process and to a makeup kit comprising the product.

This application claims benefit of U.S. Provisional Application No. 60/605,575, filed Aug. 31, 2004, and U.S. Provisional Application No. 60/624,550, filed Nov. 4, 2004, the contents of which are incorporated herein by reference. This application also claims benefit of priority under 35 U.S.C. § 119 to French Patent Application No. 04 51896, filed Aug. 24, 2004, and French Patent Application No. 04 11209, filed Oct. 21, 2004, the contents of which are also incorporated herein by reference.

The present disclosure relates to a cosmetic care or makeup composition for the skin that can have improved staying-power properties while at the same time having at least one property chosen from satisfactory migration-resistance, non-run and non-tack properties.

The cosmetic product comprises at least two compositions that may be applied successively to the skin, the eyelids, the lips and integuments, for instance the nails, the eyebrows, the eyelashes or the hair. For instance, the cosmetic product may be a foundation, a makeup rouge, an eyeshadow, a concealer product, a blusher, a lipstick, a lip balm, a lip gloss, a lip pencil, an eye pencil, a mascara, an eyeliner, a nail varnish, a body makeup product or a skin coloring product.

Transfer-resistant compositions (for instance as disclosed in U.S. Pat. No. 6,074,654, and International Patent Application Publication No. WO 02/067877) which are generally based on silicone resins and on volatile silicone oils are known in the art. Although these may have improved staying-power properties, they may also have the drawback of leaving on the skin and the lips, after evaporation of the volatile silicone oils, a film that becomes uncomfortable over time. (sensation of drying-out and of tautness), and which may deter certain users from using this type of lipstick.

It has also been proposed to apply to a first composition containing a dispersion of particles of acrylic film-forming polymer, and a second composition comprising a mixture of a phenyl silicone oil with a viscosity of 20 cSt and a phenyl silicone oil with a viscosity of 10,000 cSt (see, for example, European Patent Application No. EP 1 249 223). However, this composition can have the drawback of migrating and of becoming tacky. In addition, the cosmetic product described in the aforementioned publication does not always have sufficient staying power.

In International Patent Application Publication No. WO-A-97/17057, a method for increasing the staying-power and transfer-resistance properties is described, which comprises applying two compositions one over the other. These two compositions satisfy the following physicochemical criteria:
the composition applied first has a global Hildebrand solubility parameter of less than 8.5 (cal/cm³)^{1/2}, and
the second composition contains oils with a calculated partition coefficient ClogP of at least 13.
   However, the compositions given as examples can produce an uncomfortable makeup product.

International Patent Application Publication No. WO 02/067877 discloses a method for improving the aesthetic properties of a transfer-resistant composition, which comprises applying to a film of transfer-resistant composition, a second composition that does not destroy the film of the first composition. However, the products described in this publication can have an unpleasant odour and can be tacky.

Finally, U.S. Pat. No. 6,001,374 proposes a lip makeup, which comprises applying a base composition containing an alcohol-soluble and water-insoluble resin, onto which is deposited a composition containing a silicone compound. The silicone compounds cited as examples, for instance polydimethylsiloxane of viscosity 20,000,000 cSt and of molecular weight 500,000, as a solution in a polydimethylsiloxane of viscosity 5 cSt and of molecular weight 800, in 15/85 mass proportions (product sold under the reference SF 1236), can be too tacky, however.

Thus, the present disclosure relates to a cosmetic product, such as a makeup product, comprising at least two compositions to be applied one over the other, the product being such that the staying power is improved and the migration-resistance and non-tack properties are satisfactory. The present disclosure also relates to a cosmetic product that can have satisfactory staying power and can have improved non-tack properties.

More specifically, in at least one embodiment, the present disclosure relates to a cosmetic product, such as a makeup product, comprising at least one first composition and at least one second composition intended to be applied onto the at least one first composition, wherein the at least one first composition comprises a physiologically acceptable medium, and the at least one second composition comprises at least one first silicone polymer with a viscosity, measured according to standard ASTM D-445, ranging from 5 to 10,000 cSt, for instance from 10 to 10,000 cSt.

The at least one first composition can comprise at least one acrylic polymer. It can also be solid. As used herein, the term “solid composition” is understood to mean a stick in the case of a lipstick or a foundation.

As used herein, the term “makeup product” is understood to mean a product comprising at least one coloring agent for depositing a color onto a human keratin material (such as the skin, the lips or the integuments) by applying onto the keratin material products such as lipsticks, makeup powders, eyeliners, foundations or self-tanning products, and semi-permanent makeup products (such as tattoos).

As used herein, the term “silicone polymer” is understood to mean a polymer obtained from at least one monomer comprising at least one silicon atom.

A deposit of the product according to the present disclosure resulting from the successive application of the at least one first and at least one second compositions can have a staying-power value of greater than or equal to 60%, for example 65%, or even 70%.

The staying power is measured according to the following method:

Three glass slides covered with a sheet of collagen are prepared as follows:

A sheet of collagen (Nippi casing Grade) of 4.6 cm by 8.5 cm side length is prepared at a temperature of 28° C. and conditioned for 3 hours at 90% relative humidity (RH). The sheet of collagen is returned to the open air and is immediately fixed firmly and entirely onto a 4.6 cm by 7.6 cm glass slide. The sheet of collagen is attached to the reverse of the slide with 3M Scotch® tape. The surface of the collagen should be flat and free of folds. Each slide is left under ambient conditions for 24 hours, before performing the test. A rectangle of 4.6 cm by 7.6 cm is cut out of a Styrofoam plate (of the type Amoco Selectables Plastics DL Tableware) using a knife and a ruler by following the contour of a glass slide.

A product according to the present disclosure is applied to each glass slide and to the Styrofoam rectangle as follows: The at least one first composition is applied using a 25 μm mechanical applicator (bar coater). The at least one first composition is left to dry for 30 minutes and the at least one second composition is then applied onto the deposit of the at least one first composition.

The product is applied to the glass slides covered with collagen by applying the at least one first composition using a 25 μm mechanical applicator (bar coater). The at least one first composition is left to dry for 30 minutes and the at least one second composition is then applied onto the deposit of the at least one first composition. The plates are left under ambient conditions for 30 minutes.

Three drops of oil (about 0.075 g), spread using a brush, are applied to each of the three collagen-coated slides. The excess is blotted up with a sheet of Kimwipes paper and the slides are left for 30 minutes at room temperature. Three white Styrofoam discs 3.8 cm in diameter are cut out.

The white Styrofoam disc is firmly attached with double-sided tape to the end of a 2 kg mass and, while exerting a pressure of 175 g/cm², the weight is placed gently on the surface of a plate (product side) and the weight is rotated about itself one and a half times over a period of time ranging from 3 to 5 seconds, while maintaining the initial pressure. The weight is lifted and the Styrofoam disc is recovered. The measurement is performed for each glass slide with a clean Styrofoam disc.

The percentage of reflectance is then measured:
for the deposit of product applied to the rectangular Styrofoam sample (reference A),
for the clean white Styrofoam disc (reference B), and
for the disc detached from the weight after having applied the pressure to the slide coated with cosmetic product (reference C).
   The reflectance is measured over a wavelength range ranging from 400 nm to 700 nm using a spectral analyser (aperture 25 mm in diameter) with a D65 illuminant/10 degrees. The wavelength of the reflectance minimum is chosen for the “marked” disc. At this wavelength, the staying power is calculated according to the equation:
   100*(1−[(C−B)/(A−B)])
   The staying power is equal to the mean of the three measurements.

The present disclosure also relates to a cosmetic product, such as a makeup product, comprising at least one first composition and at least one second composition intended to be applied onto the at least one first composition, wherein the at least one first composition comprises a physiologically acceptable medium, and the at least one second composition comprises at least one silicone polymer with a viscosity, measured according to standard ASTM D-445, ranging from 5 to 10,000 cSt, the at least one silicone polymer being such that when it is in sufficient amount in the at least one second composition, the ratio between i) the staying power of a deposit of the product resulting from the successive application of the at least one first and at least one second compositions and ii) the staying power of a deposit of the at least one first composition, expressed as (i/ii) is greater than 1.1, and wherein the staying power of a deposit of the at least one first composition is greater than or equal to 30%.

In one embodiment of the present disclosure, the at least one first composition may comprise at least one acrylic polymer. In another embodiment of the present disclosure, the at least one first composition may be solid.

The ratio (i/ii) between i) the staying power of a deposit of the product resulting from the successive application of the at least one first and at least one second compositions and ii) the staying power of a deposit of the at least one first composition can be greater than or equal to 1.12, for example 1.15, or even 1.17, or even 1.20.

According to several embodiments of the present disclosure, the staying power of the at least one first composition may be greater than or equal to 40%, or even 50%, or even 55%, such as 60%.

The staying power is measured according to the method described above.

The present disclosure further relates to a cosmetic product, such as a makeup product, comprising at least one first composition and at least one second composition intended to be applied onto the at least one first composition, wherein the at least one first composition comprises a physiologically acceptable medium, and the at least one second composition comprises at least at least one first silicone polymer chosen from polydimethylsiloxanes with a viscosity, measured according to standard ASTM D-445, ranging from 5 cSt to 10,000 cSt, such as ranging from 10 cSt to 10,000 cSt, and at least one second silicone polymer chosen from polydimethylsiloxanes with a viscosity, measured according to standard ASTM D-445, ranging from 5,000 cSt to 300,000 cSt, and wherein the viscosity of the at least one second silicone polymer is greater than the viscosity of the at least one first silicone polymer.

Again, according to one embodiment of the present disclosure, the at least one first composition may comprise at least one acrylic polymer, and it may also be solid.

The present disclosure still further relates to a cosmetic product, such as a makeup product, comprising at least one first composition and at least one second composition intended to be applied onto the at least one first composition, wherein the at least one first composition comprises a physiologically acceptable medium, and the at least one second composition comprises at least one first silicone polymer with a viscosity, measured according to standard ASTM D-445, ranging from 50 cSt to 1,000 cSt, and at least one second silicone polymer with a viscosity, measured according to standard. ASTM D-445, ranging from 30,000 cSt to 300,000 cSt.

Again, according to one embodiment of the present disclosure, the at least one first composition may comprise at least one acrylic polymer, and it may also be solid.

The present disclosure additionally relates to a cosmetic product, such as a makeup product, comprising at least one first composition and at least one second composition intended to be applied onto the at least one first composition, wherein the at least one first composition comprises a physiologically acceptable medium, and the at least one second composition comprising at least one first silicone polymer with a viscosity, measured according to standard ASTM D-445, ranging from 5 cSt to 10,000 cSt, the at least one silicone polymer being such that when it is in sufficient amount in the second composition, the staying power of a deposit of the product resulting from the successive application of the at least one first and at least one second compositions is greater than 65%.

Again, the at least one first composition may comprise at least one acrylic polymer, and it may also be solid.

The present disclosure still further relates to a process for making up the skin and/or the lips and/or the integuments, comprising applying to the skin, the lips and/or the integuments at least one first coat of at least one first composition comprising a physiologically acceptable medium, and applying onto all or part of the at least one first coat, at least one second coat of at least one second composition comprising at least one silicone polymer with a viscosity, measured according to standard ASTM D-445, ranging from 5 cSt to 10,000 cSt, for instance from 10 cSt to 10,000 cSt, and wherein the staying power of a deposit resulting from the successive application of the at least one first composition and the at least one second composition is greater than 65%.

Again, the at least one first composition may comprise at least one acrylic polymer, and it may also be solid.

The present disclosure also relates to the use of at least one second composition comprising at least one silicone polymer with a viscosity, measured according to standard ASTM D-445, ranging from 5 cSt to 10,000 cSt, for instance from 10 cSt to 10,000 cSt, which is intended to be applied onto at least one first composition comprising a physiologically acceptable medium, to obtain a cosmetic product, wherein the product results in a deposit from the successive application of the at least one first and the at least one second composition, wherein the deposit has a staying power of greater than 65%.

Again, the at least one first composition may comprise at least one acrylic polymer, and it may also be solid.

The At Least One Second Composition

The at least one second composition comprises at least one silicone polymer with a viscosity, measured according to standard ASTM D-445, ranging from 10 cSt to 10,000 cSt.

The at least one second composition can have, for example, a viscosity at 25° C., measured with a Brookfield RV rotary viscometer, ranging from 3 Pa·s to 25 Pa·s.

The dynamic viscosity of the composition is measured with a Brookfield RV viscometer, which may be equipped with various spindles as a function of the order of magnitude of the viscosity that it is desired to measure. A sample composition is poured into a 600 ml beaker, which is thermostatically maintained at 25° C. for 2 hours. The measurement, given in centipoises, is taken after 10 minutes of shear. The spin speed of the spindle is 200 s⁻¹.

The density of the composition is also measured, using a pycnometer at 25° C., to convert the viscosity initially measured in centipoises into Pa·s.

According to one embodiment of the present disclosure, the at least one second composition comprises only one silicone polymer with a viscosity ranging from 10 cSt to 10,000 cSt, for instance from 50 cSt to 5,000 cSt, such as from 100 cSt to 1,000 cSt, wherein its viscosity at 25° C., measured with a Brookfield RV rotary viscometer, ranges from 3 Pa·s. to 6 Pa·s.

The inventors have discovered that using at least one second composition with a viscosity ranging from 3 Pa·s to 6 Pa·s, comprising a single polymer with a viscosity ranging from 10 cSt to 1 0,000 cSt, increases the staying power and reduces the tack of the cosmetic product according to the present disclosure.

According to another embodiment of the present disclosure, the at least one second composition comprises at least two silicone polymers with different viscosities ranging from 10 cSt to 300,000 cSt.

For example, the at least one second composition can comprise at least one first silicone polymer with a viscosity ranging from 10 cSt to 10,000 cSt, and at least one second silicone polymer with a viscosity greater than that of the at least one first silicone polymer and ranging from 5,000 cSt to 300,000 cSt.

The at least one second silicone polymer has a weight-average molar mass of less than 200,000 g/mol. The molecular mass of the silicone compound is measured by gel permeation chromatography (GPC).

In this embodiment, the viscosity at 25° C. of the at least one second composition can range, for example, from 5 Pa·s to 25 Pa·s, such as from 8 Pa·s to 15 Pa·s. The inventors have discovered that the choice of two polymers with the specific viscosities makes it possible to improve the staying power and to reduce the tack of the cosmetic product according to the present disclosure.

According to another embodiment, the at least one second composition is transparent. As used herein, the term “transparent composition” is understood to mean a composition that is transparent to translucent, i.e. it transmits at least 40% of light, or for example, at least 50% of light at a wavelength of 750 nm. The transmission measurement is performed using a Cary 300 Scan UV-visible spectrophotometer from the company Varian according to the following protocol:

A sample of the composition is poured above its melting point into a spectrophotometer cuvette of square cross-section with a side length of 10 mm. The sample of the composition is then cooled for 24 hours at 35° C., and then maintained in a chamber thermostatically maintained at 20° C. for 24 hours.

The light transmitted through the sample of the composition is then measured using the spectrophotometer by scanning with wavelengths ranging from 700 nm to 800 nm, the measurement being performed in transmission mode.

The percentage of light transmitted through the sample of the composition at a wavelength of 750 nm is then determined.

The at least one first silicone polymer with a viscosity ranging from 10 cSt to 10,000 cSt may be chosen from those of formula (A): 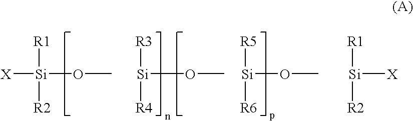
wherein:
R₁, R₂, R₅ and R₆, which may be identical or different, are chosen from alkyl radicals comprising from 1 to 6 carbon atoms, optionally substituted with at least one fluorine atom,
R₃ and R₄, which may be identical or different, are chosen from alkyl radicals comprising from 1 to 6 carbon atoms and aryl radicals,
X is chosen from alkyl radicals comprising from 1 to 6 carbon atoms, hydroxyl radicals, vinyl radicals, allylic radicals, and alkoxy radicals comprising from 1 to 6 carbon atoms,
n and p are chosen such that the silicone polymer has a viscosity at 25° C., measured according to standard ASTM D-445, ranging from 10 cSt to 10,000 cSt.
   In one embodiment of the present disclosure, p is equal to 0.

The at least one silicone polymer with a viscosity ranging from 10 cSt to 10,000 cSt may also be chosen from polyalkylsiloxanes, polyarylsiloxanes and polyalkylarylsiloxanes.

The at least one silicone polymer with a viscosity ranging from 10 cSt to 10,000 cSt may further be chosen from, by way of non-limiting example:
polydimethylsiloxanes (PDMS);
polydimethylsiloxanes comprising alkyl, alkoxy or phenyl groups, which are pendent or at the end of a silicone chain, these groups comprising from 2 to 24 carbon atoms;
phenyl silicones, for instance phenyl trimethicones, phenyl dimethicones, phenyl trimethylsiloxy diphenylsiloxanes, diphenyl dimethicones, diphenyl methyldiphenyl trisiloxanes and 2-phenylethyl trimethylsiloxysilicates.

Among the polyalkylsiloxanes that may be used according to the present disclosure, non-limiting examples include polydimethylsiloxanes, (polydimethylsiloxane)/(methylvinylsiloxane) copolymers, poly(dimethylsiloxane)(diphenyl)siloxanes and poly(dimethylsiloxane)(diphenyl)(methylvinylsiloxane) copolymers, and mixtures thereof.

The at least one silicone polymer with a viscosity ranging from 10 cSt to 10,000 cSt, in at least one embodiment, is a polydimethylsiloxane.

The at least one silicone polymer with a viscosity ranging from 10 cSt to 10,000 cSt can have, for example, a viscosity, measured according to standard ASTM D-445, ranging from 50 cSt to 5,000 cSt, for instance from 100 cSt to 3,000 cSt, such as from 100 cSt to 1,000 cSt, for example equal to 350 cSt.

According to another embodiment of the present disclosure, the at least one second composition comprises at least one first silicone polymer with a viscosity ranging from 10 cSt to 10,000 cSt, and at least one second silicone polymer with a viscosity greater than that of the first silicone polymer and ranging from 5,000 cSt to 300,000 cSt, the viscosities being measured according to standard ASTM D-445.

The at least one second silicone polymer can have, for example, a viscosity ranging from 5,000 cSt to 300,000 cSt, such as from 10,000 cSt to 200,000 cSt, for example from 30,000 to 100,000 cSt.

For instance, the at least one first silicone polymer with a viscosity ranging from 10 cSt to 10,000 cSt can have a viscosity ranging from 10 cSt to 5,000 cSt, for example from 10 cSt to 1,000 cSt, such as from 10 cSt to 500 cSt, and the at least one second silicone polymer with a viscosity greater than that of the first silicone polymer can range from 5,000 cSt to 300,000 cSt, such as ranging from 10,000 to 300,000 cSt.

The mass proportion between the at least one first and at least one second silicone polymers can be adjusted, for example, according to the viscosity of the second composition that it is desired to obtain, so as to optimize the staying power and the non-tacky nature of the cosmetic product according to the present disclosure.

The at least one second silicone polymer, which has a viscosity ranging from 5,000 cSt to 300,000 cSt may be chosen from those of formula (B): 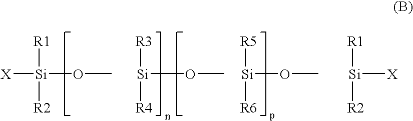
wherein:
R₁, R₂, R₅ and R₆, which may be identical or different, are chosen from alkyl radicals comprising from 1 to 6 carbon atoms, optionally substituted with at least one fluorine atom,
R₃ and R₄, which may be identical or different, are chosen from alkyl radicals comprising from 1 to 6 carbon atoms and aryl radicals,
X is chosen from alkyl radicals comprising from 1 to 6 carbon atoms, hydroxyl radicals, vinyl radicals, allylic radicals, and alkoxy radicals comprising from 1 to 6 carbon atoms,
n and p are chosen such that the at least one second silicone polymer has a viscosity at 25° C., measured according to standard ASTM D-445, ranging from 5,000 cSt to 300,000 cSt.
   In at least one embodiment of the present disclosure, p is equal to 0.

The at least one second silicone polymer, which has a viscosity ranging from 5,000 cSt to 300,000 cSt, may also be chosen from polyalkylsiloxanes, polyarylsiloxanes and polyalkylarylsiloxanes.

The at least one second silicone polymer, which has a viscosity ranging from 5,000 cSt to 300,000 cSt may further be chosen from, by way of non-limiting example:
polydimethylsiloxanes (PDMS);
polydimethylsiloxanes comprising alkyl, alkoxy or phenyl groups, which are pendent or at the end of a silicone chain, these groups coing from 2 to 24 carbon atoms;
phenyl silicones, for instance phenyl trimethicones, phenyl dimethicones, phenyl trimethylsiloxy diphenylsiloxanes, diphenyl dimethicones, diphenyl methyldiphenyl trisiloxanes and 2-phenylethyl trimethylsiloxysilicates.

Among the polyalkylsiloxanes according to the present disclosure, non-limiting mention may be made of polydimethylsiloxanes, (polydimethylsiloxane)/(methylvinylsiloxane) copolymers, poly(dimethylsiloxane)(diphenyl)siloxanes and poly(dimethylsiloxane)(diphenyl)(methylvinylsiloxane) copolymers, and mixtures thereof.

In one embodiment of the present disclosure, the at least one second silicone polymer, which has a viscosity ranging from 5,000 cSt to 300,000 cSt, is a polydimethylsiloxane.

In another embodiment of the present disclosure, the at least two silicone polymers of different viscosities are both polydimethylsiloxanes.

According to still another embodiment of the present disclosure, the at least one second composition comprises at least one first polydimethylsiloxane with a viscosity ranging from 50 cSt to 5,000 cSt, and at least one second polydimethylsiloxane with a viscosity ranging from 10,000 cSt to 300,000 cSt, the viscosities being measured according to standard ASTM D-445.

In this embodiment, for example, the proportion of the at least two polydimethylsiloxanes can be chosen such that the viscosity of the at least one second composition ranges from 5 Pa·s to 25 Pa·s.

The at least one first silicone polymer for example has a viscosity, measured at 25° C. according to standard ASTM D-445, ranging from 50 cSt to 1000 cSt, for instance from 100 cSt to 500 cSt, and the at least one second silicone polymer can have a viscosity, for example, measured at 25° C. according to standard ASTM D-445, ranging from 30,000 cSt to 300,000 cSt, such as from 30,000 cSt to 250,000 cSt.

The at least two silicone polymers may be chosen from the polymers described above.

The at least one second composition can comprise, for example, less than 50% by weight of a silicone oil with a viscosity at 25° C., measured according to standard ASTM D-445, of less than 50 cSt, such as less than or equal to 20 cSt, for instance, less than or equal to 10 cSt, such as less than or equal to 5 cSt. The at least one second composition for example, can comprise less than 40%, such as less than or equal to 30% and less than or equal to 20% by weight of this silicone oil, the viscosity of which may be greater than or equal to 0.01 cSt.

The at least one second composition can comprise, for example, less than 10% by weight of pigments and/or fillers as defined below. For instance, the at least one second composition can comprise less than or equal to 5% by weight of pigments and/or fillers.

Silicone Polymer of Very High Viscosity

The at least one second composition may also optionally comprise at least one silicone polymer with a viscosity greater than 300,000 cSt, or at least one solid silicone polymer optionally provided in solubilized form in a solvent. This silicone polymer can be for example, a non-grafted polymer, i.e. a polymer obtained by polymerization of at least one monomer, without subsequent reaction of the side chains with another chemical compound. The silicone polymer can be chosen, for instance, from dimethiconols, fluorosilicones and dimethicones, and mixtures thereof.

According to one embodiment of the present disclosure, the at least one silicone polymer with a viscosity of greater than 300,000 cSt is the dimethiconol sold by Dow Corning under the reference D2-9085, or the dimethiconol sold by Dow Corning under the reference DC 1503. The dimethiconol sold by Dow Corning under the reference Q2-1403 or Q2-1401 may also be chosen.

Among the silicone polymers with a viscosity of greater than 300 000 cSt, non-limiting mention may also be made of SE30, SE33, SE 54 and SE 76 sold by the company General Electric, AK 500000 sold by the company Wacker, and Silbione 70047 V sold by the company Rhodia.

Further non-limiting examples of the dimethicones according to the present disclosure include polydimethylsiloxanes, (polydimethylsiloxane)/(methylvinylsiloxane) copolymers and poly(dimethylsiloxane)(diphenyl)(methylvinylsiloxane) copolymers, and mixtures thereof.

The at least one second composition may comprise the at least one silicone polymer with a viscosity of greater than 300,000 cSt or the at least one solid silicone polymer in an amount ranging from, for example, 0.1% to 20% by weight, such as from 0.1% to 5% by weight.

Volatile Oil

The at least one second composition may optionally comprise at least one volatile oil.

As used herein, the term “volatile oil” is understood to mean an oil (or non-aqueous medium) capable of evaporating on contact with the skin in less than one hour, at room temperature and atmospheric pressure. The volatile oil is a volatile cosmetic oil, which is liquid at room temperature, such as having a non-zero vapour pressure, at room temperature and atmospheric pressure, for instance having a vapour pressure ranging from 0.13 Pa to 40,000 Pa (10⁻³ mmHg to 300 mmHg), for example ranging from 1.3 Pa to 13,000 Pa (0.01 mmHg to 100 mmHg), such as ranging from 1.3 Pa to 1,300 Pa (0.1 mmHg to 10 mmHg).

In addition, the volatile oil can have a boiling point, measured at atmospheric pressure, ranging from 150° C. to 260° C., such as ranging from 170° C. to 250° C.

As used herein, the term “hydrocarbon-based oil” is understood to mean an oil formed essentially from, or even consisting of, carbon and hydrogen atoms, and possibly oxygen and nitrogen atoms, and comprising no silicon or fluorine atoms; it may also comprise ester, ether, amine or amide groups.

As used herein, the term “silicone oil” is understood to mean an oil comprising at least one silicon atom, such as comprising Si—O groups.

The at least one optional volatile oil may be a silicone oil or a hydrocarbon-based oil.

The at least one volatile silicone oil that may be used in the present disclosure may be chosen from silicone oils with a flash point ranging from 40° C. to 102° C., for instance with a flash point ranging from 55° C. to 95° C., such as ranging from 65° C. to 95° C.

Among the volatile silicone oils that may be used in the present disclosure, non-limiting mention may be made of linear or cyclic silicones with a viscosity at room temperature of less than 8 cSt and for example, comprising from 2 to 7 silicon atoms, these silicones optionally comprising alkyl or alkoxy groups comprising from 1 to 10 carbon atoms. Among the volatile silicone oils that may be used in the present disclosure, non-limiting mention may be made of, for example, octamethylcyclotetrasiloxane, decamethylcyclopentasiloxane, dodecamethylcyclohexasiloxane, heptamethylhexyltrisiloxane, heptamethyloctyltrisiloxane, octamethyltrisiloxane and decamethyltetrasiloxane, and mixtures thereof.

Further among the volatile silicone oils that may be used in the present disclosure, non-limiting mention may also be made of the silicones described in French Patent Application Publication No. FR 2 853 227 (French Patent Application No. FR 03/04259).

The volatile hydrocarbon-based oil that may be used in the present disclosure may be chosen from hydrocarbon-based oils with a flash point ranging from 40° C. to 102° C., for instance ranging from 50° C. to 100° C., such as ranging from 75° C. to 85° C.

Volatile hydrocarbon-based oils that may be mentioned include, by way of non-limiting example, volatile hydrocarbon-based oils comprising from 8 to 16 carbon atoms and mixtures thereof, for example branched C₈-C₁₆ alkanes, for instance C₈-C₁₆ isoalkanes (also known as isoparaffins), isododecane, isodecane and isohexadecane and, for example, the oils sold under the trade names Isopar or Permethyl, branched C₈-C₁₆ esters, for instance isohexyl neopentanoate, and mixtures thereof. For example, the volatile hydrocarbon-based oil can be chosen from volatile hydrocarbon-based oils comprising from 8 to 16 carbon atoms and mixtures thereof, such as from isododecane, isodecane and isohexadecane. In one embodiment of the present disclosure, the volatile hydrocarbon-based oil is isododecane.

The at least one volatile oil, when present, can be present in the at least one second composition in an amount ranging from 0.1% to 50% by weight, relative to the total weight of the at least one second composition, for instance from 1% to 30% by weight, such as from 5% to 15% by weight, relative to the total weight of the at least one second composition.

Gelling Agent

According to one embodiment of the present disclosure, the at least one second composition may also comprise at least one gelling agent, which is a polymeric or mineral gelling agent.

As used herein, the term “gelling agent” is understood to mean a compound that increases the viscosity of the medium into which it is incorporated, or which rigidities the medium. The gelling agent according to the present disclosure does not include waxes.

According to another embodiment of the present disclosure, the gelling agent may be chosen from, by way of non-limiting example:
a fumed silica optionally hydrophobic-surface-treated; and
optionally modified clays, for instance hectorites modified with a C₁₀ to C₂₂ fatty acid ammonium chloride, for instance hectorite modified with distearyldimethylammonium chloride, and mixtures thereof.

Gelling agents that may also be mentioned in a non-limiting manner include partially or totally crosslinked elastomeric organopolysiloxanes, of three-dimensional structure, for instance those sold under the names KSG6, KSG16 and KSG18 from Shin-Etsu, Trefil E-505C or Trefil E-506C from Dow Corning, Gransil SR-CYC, SR DMF10, SR-DC556, SR 5CYC Gel, SR DMF 10 Gel and SR DC 556 Gel from Grant Industries, and SF 1204 and JK 113 from General Electric.

Wax

The at least one second composition may optionally comprise at least one wax.

The at least one wax may be present in an amount ranging from 2% to 30% by weight, relative to the total weight of the at least one second composition, for instance ranging from 5% to 20% by weight, such as ranging from 5% to 15% by weight, relative to the total weight of the at least one second composition.

In one embodiment, linear hydrocarbon-based waxes are used in the at least one second composition according to the present disclosure. Their melting point can be for instance, greater than 35° C., for example greater than or equal to 55° C., such as greater than or equal to 80° C.

The linear hydrocarbon-based waxes can be chosen, by way of non-limiting example, from substituted linear alkanes, unsubstituted linear alkanes, unsubstituted linear alkenes and substituted linear alkenes, and an unsubstituted compound being a compound of carbon and hydrogen exclusively. The substituents mentioned above do not comprise any carbon atoms.

The linear hydrocarbon-based waxes include ethylene polymers and copolymers with a molecular weight of between 400 and 800, for example Polywax 500 or Polywax 400 sold by New Phase Technologies.

The linear hydrocarbon-based waxes also include linear paraffin waxes, for instance the paraffins S&P 206, S&P 173 and S&P 434 from Strahl & Pitsch.

The linear hydrocarbon-based waxes further include long-chain linear alcohols, for instance products comprising a mixture of polyethylene and of alcohols comprising 20 to 50 carbon atoms, such as Performacol 425 or Performacol 550 (mixture in 20/80 proportions) sold by New Phase Technologies.

The at least one second composition can comprise, for example, a silicone wax, such as a dimethicone comprising alkyl groups at the end of the chain. These alkyl groups can comprise, for example, more than 18 carbon atoms, for instance from 20 to 50 carbon atoms, such as from 30 to 45 carbon atoms. Non-limiting examples of silicone waxes that may be used include:
C₂₀₋₂₄ alkyl methicones, C₂₄₋₂₈ alkyl dimethicones, C₂₀₋₂₄ alkyl dimethicones and C₂₄₋₂₈ alkyl dimethicones sold by Archimica Fine Chemicals under the reference SilCare 41M40, SilCare 41M50, SilCare 41M70 and SilCare 41M80,
stearyl dimethicones of reference SilCare 41M65 sold by Archimica or of reference PC-2503 sold by Dow Corning,
stearoxytrimethylsilanes sold under the reference SilCare 1M71 or DC-580,
the products Abil Wax 9810, 9800 or 2440 from Wacker-Chemie GmbH,
C₃₀₋₄₅ alkyl methicones sold by Dow Corning under the reference AMS-C30 Wax, and also the C₃₀₋₄₅ alkyldimethicones sold by General Electric under the reference SF 1642 or SF 1632.

According to one embodiment of the present disclosure, the at least one second composition comprises wax in an amount less than 5% by weight, such as less than or equal to 3%, or even less than or equal to 1% by weight, relative to the total weight of the at least one second composition.

The At Least One First Composition

The at least one first composition comprises a physiologically acceptable medium, i.e. a cosmetically or dermatologically acceptable medium, i.e. a non-toxic medium that may be applied to human skin, integuments or lips of the face. As used herein, the term “cosmetically acceptable” is understood to mean a composition of pleasant appearance, odor and feel.

The at least one first composition may be any type of cosmetic composition known to those skilled in the art. For example, the at least one first composition can be a cosmetic composition with good staying-power properties, such as good oil resistance.

Film-Forming Polymer

According to one embodiment, the at least one first composition according to the present disclosure can comprise, for example, at least one film-forming polymer and a liquid organic phase, such as a liquid fatty phase.

As used herein, the term “film-forming polymer” is understood to mean a polymer capable of forming, by itself or in the presence of an auxiliary film-forming agent, a continuous film that adheres to a support, such as to keratin materials, for instance a cohesive film, or for example, a film whose cohesion and mechanical properties are such that the film may be isolated from the said support.

The at least one film-forming polymer can be, for example, one of the polymers described in the French Patent Application Publication No. FR 2 863 493 (French Patent Application No. FR 04/50540), the content of which is incorporated herein by reference.

As used herein, the term “liquid fatty phase” is understood to mean any non-aqueous medium that is liquid at room temperature (25° C.) and atmosphere pressure (760 mmHg), composed of at least one fatty substance that is liquid at room temperature, also known as oils, with the exception of the ester of acid and of polyol, and also gelling agents and stabilizers for the pigments that may be present in the composition, when they are liquid at room temperature and atmospheric pressure. This fatty phase may comprise a volatile liquid fatty phase and/or a non-volatile fatty phase.

The at least one film-forming polymer, in at least one embodiment, is a non-crystalline ethylenic polymer. As used herein, the term “ethylenic polymer” is understood to mean a polymer obtained by polymerization of ethylenically unsaturated monomers.

The at least one film-forming polymer can be the form of a dispersion of particles in a liquid fatty phase.

According to one embodiment, the at least one first composition comprises a stable dispersion of generally spherical polymer particles of at least one polymer, in a physiologically acceptable liquid organic phase.

These dispersions may be, for example, in the form of polymer nanoparticles in stable dispersion in the liquid organic phase. The nanoparticles can have a mean size, for instance, ranging from 5 nm to 800 nm, such as from 50 nm to 500 nm. However, it is possible for the dispersions to comprise polymer particles ranging up to 1 μm in size.

These polymer particle dispersions can be, by way of non-limiting example, those described in European Patent Application Publication No. EP 0 749 747, the content of which is incorporated herein by reference.

For example, the polymer particles in dispersion can be insoluble in water-soluble alcohols, for instance ethanol.

The polymers in dispersion that may be used in the at least one first composition of the present disclosure can have, for example, a molecular weight ranging from 2,000 g/mol to 10,000,000 g/mol, and a Tg ranging from −100° C. to 300° C., for instance ranging from −50° C. to 100° C., such as from −10° C. to 50° C.

When a polymer has a glass transition temperature that is too high for the intended application, a plasticizer may be combined therewith so as to lower this temperature of the mixture used.

It is possible to use polymers having a low Tg, for example, which is less than or equal to skin temperature, for instance less than or equal to 40° C., such as ranging from −10° C. to 30° C.

As used herein, the expression “free-radical polymer” is understood to mean a polymer obtained by polymerization of monomers comprising unsaturation, such as ethylenic unsaturation, each monomer being capable of homopolymerizing (unlike polycondensates). The free-radical polymers may also be, for example, vinyl polymers or copolymers, such as acrylic polymers.

The acrylic polymers may result from the polymerization of ethylenically unsaturated monomers comprising at least one acid group and/or esters of these acidic monomers and/or amides of these acids.

Monomers bearing an acidic group that may be used include α,β-ethylenic unsaturated carboxylic acids such as acrylic acid, methacrylic acid, crotonic acid, maleic acid and itaconic acid. For example, in one embodiment, (meth)acrylic acid and crotonic acid are used, and in another embodiment, (meth)acrylic acid is used.

The esters of acidic monomers can be chosen from, by way of non-limiting example, (meth)acrylic acid esters (also known as (meth)acrylates), for instance (meth)acrylates of an alkyl, such as of a C₁-C₂₀ and C₁-C₈ alkyl, (meth)acrylates of an aryl, for instance of a C₆-C₁₀ aryl, (meth)acrylates of a hydroxyalkyl, such as of a C₂-C₆ hydroxyalkyl. Alkyl(meth)acrylates that may be mentioned in a non-limiting manner include methyl, ethyl, butyl, isobutyl, 2-ethylhexyl and lauryl(meth)acrylate. Hydroxyalkyl(meth)acrylates that may be mentioned in a non-limiting manner include hydroxyethyl(meth)acrylate and 2-hydroxypropyl(meth)acrylate. Aryl(meth)acrylates that may be mentioned in a non-limiting manner include benzyl or phenyl acrylate.

The (meth)acrylic acid esters that may be used include, for example, alkyl (meth)acrylates.

Free-radical polymers that can be used by way of non-limiting example, include copolymers of (meth)acrylic acid and of an alkyl(meth)acrylate, such as of a C₁-C₄ alkyl. Further non-limiting mention may be made of methyl acrylates optionally copolymerized with acrylic acid.

Amides of the acidic monomers that may be mentioned in a non-limiting manner include (meth)acrylamides, for instance N-alkyl(meth)acrylamides, for example of a C₂-C₁₂ alkyl, such as N-ethylacrylamide, N-t-butylacrylamide and N-octylacrylamide; N-di(C₁-C₄)alkyl(meth)acrylamides.

The acrylic polymers may also result from the polymerization of ethylenically unsaturated monomers comprising at least one amine group, in free form or in partially or totally neutralized form, or alternatively in partially or totally quaternized form. Such monomers may be, by way of non-limiting example, dimethylaminoethyl(meth)acrylate, dimethylaminoethylmethacrylamide, vinylamine, vinylpyridine or diallyldimethylammonium chloride.

The vinyl polymers may also result from the homopolymerization or copolymerization of at least one monomer chosen from vinyl esters and styrene monomers. For instance, these monomers may be polymerized with acidic monomers and/or esters thereof and/or amides thereof, such as those mentioned above. Non-limiting examples of vinyl esters that may be mentioned include vinyl acetate, vinyl propionate, vinyl neodecanoate, vinyl pivalate, vinyl benzoate and vinyl t-butylbenzoate. Styrene monomers that may be mentioned in a non-limiting manner include styrene and α-methylstyrene.

The list of monomers given above is not limiting and it is possible to use any monomer known to those skilled in the art falling within the categories of acrylic and vinyl monomers (including monomers modified with a silicone chain).

Among other vinyl monomers that may be used, non-limiting mention may also be made of:
N-vinylpyrrolidone, vinylcaprolactam, vinyl-N-(C₁-C₆)alkylpyrroles, vinyloxazoles, vinylthiazoles, vinylpyrimidines and vinylimidazoles, and
olefins such as ethylene, propylene, butylene, isoprene and butadiene.

The vinyl polymer may be crosslinked by means of at least one difunctional monomer comprising, for example, at least two ethylenic unsaturations, such as ethylene glycol dimethacrylate or diallyl phthalate.

In a non-limiting manner, the polymers in dispersion of the present disclosure may be chosen from the following polymers or copolymers: polyurethanes; polyurethane-acrylics; polyureas; polyurea-polyurethanes; polyester-polyurethanes; polyether-polyurethanes; polyesters; polyesteramides; fatty-chain polyesters; alkyds; acrylic and/or vinyl polymers or copolymers; acrylic-silicone copolymers; polyacrylamides; silicone polymers, for instance silicone acrylics or polyurethanes, fluoro polymers, and mixtures thereof.

The at least one polymer in dispersion in the organic liquid phase can be present as solids in an amount ranging from 2% to 40%, for instance from 5% to 40%, such as from 5% to 35% and from 8% to 30% by weight, relative to the weight of the at least one first composition.

The at least one first composition may optionally comprise, for example, at least one stabilizer, which can be chosen from block polymers, grafted polymers and/or a random polymers. The stabilization may be performed by any known means, for example by direct addition of the block polymer, grafted polymer and/or random polymer, during the polymerization. Among the grafted-block or block copolymers that may be used, non-limiting mention may also be made of those comprising at least one block resulting from the polymerization of at least one ethylenic monomer, comprising at least one optionally conjugated ethylenic bond, for instance ethylene or dienes such as butadiene and isoprene, and of at least one block of a vinyl polymer, such as a styrene polymer. When the ethylenic monomer comprises several optionally conjugated ethylenic bonds, the residual ethylenic unsaturations after the polymerization are generally hydrogenated.

Thus, in a known manner, the polymerization of isoprene can lead, after hydrogenation, to the formation of an ethylene-propylene block, and the polymerization of butadiene can lead, after hydrogenation, to the formation of an ethylene-butylene block. Among these polymers, non-limiting mention may be made of block copolymers, especially of “diblock” or “triblock” type, such as polystyrene/polyisoprene (SI) or polystyrene/polybutadiene (SB) such as those sold under the name “Luvitol HSB” by BASF, of the polystyrene/copoly(ethylene-propylene) (SEP) type such as those sold under the name “Kraton” by Shell Chemical Co., or alternatively of the polystyrene/copoly(ethylene-butylene) (SEB) type. Kraton G1650 (SEBS), Kraton G1651 (SEBS), Kraton G1652 (SEBS), Kraton G1657X (SEBS), Kraton G1701X (SEP), Kraton G1702X (SEP), Kraton G1726X (SEB), Kraton D-1101 (SBS), Kraton D-1102 (SBS) and Kraton D-1107 (SIS) may also be mentioned in a non-limiting manner. The polymers are generally known as hydrogenated or non-hydrogenated diene copolymers.

The stabilizer can also be present in the mixture, for example, before polymerization of the polymer. However, it is also possible to add it continuously, such as when the monomers are also added continuously.

The at least one stabilizer may be present in an amount ranging from 2% to 30% by weight, such as from 5% to 20% by weight, relative to the weight of the initial monomer mixture.

In one embodiment of the present disclosure, the liquid fatty phase of the at least one first composition may comprise any cosmetically or dermatologically acceptable oil, and in general any physiologically acceptable oil, chosen from, for example, oils of mineral, plant or synthetic origin, carbon-based oils, hydrocarbon-based oils, fluoro oils and/or silicone oils, alone or as a mixture provided that they form a homogeneous and macroscopically stable mixture and that they are compatible with the intended use.

The total liquid fatty phase of the at least one first composition can be present in an amount ranging from 5% to 90% by weight, such as from 20% to 85% by weight, relative to the total weight of the at least one first composition. For example, it can be present in an amount of greater than or equal to 30% of the total weight of the at least one first composition. For further example, this fatty phase can comprise at least one volatile oil.

According to one embodiment of the present disclosure, at least one volatile oil is used.

The at least one volatile oil may be hydrocarbon-based oils or silicone oils optionally comprising alkyl or alkoxy groups that are pendent or at the end of a silicone chain.

Among the volatile silicone oils that may be used in the present disclosure, non-limiting mention may be made of linear or cyclic silicones with a viscosity at room temperature of less than 8 cSt, for instance comprising from 2 to 7 silicon atoms, these silicones optionally comprising alkyl or alkoxy groups comprising from 1 to 10 carbon atoms. Among the volatile silicone oils that may be used in the at least one first composition of the present disclosure, non-limiting mention may be made of, for example, octamethylcyclotetrasiloxane, decamethylcyclopentasiloxane, dodecamethylcyclohexasiloxane, heptamethylhexyltrisiloxane, heptamethyloctyltrisiloxane, octamethyltrisiloxane and decamethyltetrasiloxane, and mixtures thereof.

Among the other volatile oils that may be used in the at least one first composition of the present disclosure, non-limiting mention may be made of C₈-C₁₆ isoalkane oils (also known as isoparaffins), for instance isododecane, isodecane and isohexadecane and, for example, the oils sold under the trade names Isopar and Permethyl, and such as isododecane (Permethyl 99A).

The at least one volatile oil can be present for example, in an amount ranging from 5% to 85% by weight, such as from 20% to 75% by weight, relative to the total weight of the at least one first composition.

Synthesis Solvent for the Polymer Particles

The polymer dispersion may be manufactured as described in European Patent Application Publication No. EP-A-0 749 747.

A mixture comprising the initial monomers and also a free-radical initiator is prepared. This mixture is dissolved in a solvent referred to hereinbelow in the present disclosure as the “synthesis solvent”. When the fatty phase is a non-volatile oil, the polymerization may be performed in an apolar organic solvent (synthesis solvent), followed by adding the non-volatile oil (which should be miscible with the synthesis solvent) and selectively distilling off the synthesis solvent.

A synthesis solvent should be chosen such that the initial monomers and the free-radical initiator are soluble therein, and the polymer particles obtained are insoluble therein, so that they precipitate during their formation. For instance, the synthesis solvent may be chosen from alkanes such as heptane, isododecane and cyclohexane.

When the fatty phase chosen is a volatile oil, the polymerization may be performed directly in the oil, which thus also acts as synthesis solvent. The monomers should also be soluble therein, as should the free-radical initiator, and the polymer obtained should be insoluble therein.

The monomers can be present in the synthesis solvent, before polymerization, in an amount ranging from 5% to 20% by weight, relative to the reaction mixture. The total amount of monomers can be present in the solvent before the start of the reaction, or part of the monomers may be added gradually as the polymerization reaction proceeds.

The free-radical initiator may be, for example, azobisisobutyronitrile or tert-butylperoxy-2-ethyl hexanoate.

The volatile phase of the composition may comprise the synthesis solvent for the dispersed polymer particles.

Non-Volatile Oil of the Fatty Phase

The fatty phase preferably comprises at least one apolar or sparingly polar non-volatile oil.

Among the sparingly polar non-volatile oils that may be used in the at least one first composition of the present disclosure, non-limiting mention may be made of apolar oils or, for example, oils comprising an alkyl chain such as C₃-C₄₀. Non-limiting examples of apolar or sparingly polar oils that may be mentioned include:
linear or branched hydrocarbons such as liquid paraffin, liquid petroleum jelly and liquid light naphthas, and hydrogenated polyisobutene;
hydrocarbon-based oils of animal origin, for instance squalene;
hydrocarbon-based plant oils such as liquid triglycerides of fatty acids of at least 10 carbon atoms;
synthetic esters and ethers, such as fatty acids, for instance the oils of formula R₁CO(O)ₓR₂ in which R₁ is an acid residue comprising from 2 to 29 carbon atoms with x being equal to 0 or 1, and R₂ is a hydrocarbon-based chain comprising from 3 to 30 carbon atoms, for instance tributyl acetyl citrate, oleyl erucate, 2-octyldodecyl behenate, triisoarachidyl citrate, isocetyl stearoylstearate or octyldodecanyl stearoylstearate, n-propyl acetate, tridecyl trimellitate, diisocetyl dodecanedioleate or stearate, arachidyl propionate, dibutyl phthalate, propylene carbonate or octyidodecyl pentanoate; polyol esters, for instance vitamin F, sorbitan isostearate and glyceryl or diglyceryl triisostearate;
silicone oils such as polydimethylsiloxanes (PDMS), optionally comprising a C₃-C₄₀ alkyl or alkoxy chain or a phenylated chain, such as optionally fluorinated phenyl trimethicones and polyalkylmethylsiloxanes, for instance polymethyltrifluoropropyidimethylsiloxanes, or with functional groups such as hydroxyl, thiol and/or amine groups; polysiloxanes modified with fatty acids, fatty alcohols or polyoxyalkylenes,
fluoro oils; and
mixtures thereof.

These apolar or sparingly polar non-volatile oils can be present in an amount ranging from 0.1% to 20% by weight, relative to the total weight of the composition, for instance from 0.5% to 10% by weight, such as from 1% to 5% by weight, relative to the total weight of the at least one first composition.

For example, in one embodiment of the present disclosure, the at least one non-volatile oil is apolar. It may be chosen from hydrocarbons, such as alkanes, for instance hydrogenated polyisobutene.

The fatty phase may also comprise at least one polar oil chosen from fatty acid esters of 7 to 29 carbon atoms, for instance diisostearyl malate, isopropyl palmitate, diisopropyl adipate, caprylic/capric acid triglycerides such as those sold by the company Stearineries Dubois or those sold under the names Miglyol 810, 812 and 818 by the company Dynamit Nobel, shea butter oil, isopropyl myristate, butyl stearate, hexyl laurate, diisopropyl adipate, isononyl isononate, 2-hexyldecyl laurate, 2-octyidecyl palmitate, 2-octyldodecyl myristate or lactate, 2-diethylhexyl succinate, 2-ethylhexyl palmitate, 2-octyldodecyl stearate or castor oil; esters of lanolic acid, of lauric acid or of stearic acid; higher fatty alcohols (of 7 to 29 carbon atoms) such as stearyl alcohol, linoleyl alcohol, linolenyl alcohol, isostearyl alcohol, 2-octyldodecanol, decanol, dodecanol, octadecanol or oleyl alcohol; higher fatty acids (of 7 to 29 carbon atoms) such as myristic acid, palmitic acid, stearic acid, behenic acid, oleic acid, linoleic acid, linolenic acid or isostearic acid; and mixtures thereof.

The at least one non-volatile polar oil may be present in an amount ranging from 0.1% to 10%, such as from 1% to 5% by weight, relative to the total weight of the at least one first composition.

According to another embodiment of the present disclosure, the at least one liquid fatty phase comprises at least one apolar volatile oil and at least one apolar non-volatile oil. In this embodiment, the at least one first composition comprises, for example, a dispersant for the fillers and/or pigments.

For instance, 12-hydroxystearic acid esters and C₈ to C₂₀ fatty acid esters of polyols such as glycerol or diglycerol can be used, such as poly(12-hydroxystearic acid)stearate with a molecular weight of about 750 g/mol, such as the product sold under the name Solsperse 21 000 by the company Avecia, polyglyceryl-2 dipolyhydroxystearate (CTFA name) sold under the reference Dehymyls PGPH by the company Henkel, or polyhydroxystearic acid such as the product sold under the reference Arlacel P100 by the company Uniqema, and mixtures thereof.

As another dispersant that may be used in the at least one first composition of the present disclosure, non-limiting mention may be made of quaternary ammonium derivatives of polycondensed fatty acids, for instance Solsperse 17 000 sold by the company Avecia, and polydimethylsiloxane/oxypropylene mixtures such as those sold by the company Dow Corning under the references DC2-5185 and DC2-5225 C.

The polydihydroxystearic acid and the 12-hydroxystearic acid esters can be used, for example, for a hydrocarbon-based or fluorinated medium, whereas the mixtures of oxyethylene/oxypropylene dimethylsiloxane can be used, for instance, for a silicone medium.

According to another embodiment, the at least one first composition according to the present disclosure may comprise as film-forming polymer a dispersion of particles, which may be solid, of a grafted ethylenic polymer in a liquid fatty phase.

As used herein, the term “grafted polymer” is understood to mean a polymer having a skeleton comprising at least one side chain that is pendent, or located at the end of the chain, such as pendent.

For example, the grafted ethylenic polymer can comprise an ethylenic skeleton that is insoluble in the liquid fatty phase, and side chains covalently linked to the skeleton, which are soluble in the liquid fatty phase.

The grafted ethylenic polymer can be, for instance, a non-crosslinked polymer. For example, the polymer can be obtained by polymerization of monomers comprising only one polymerizable group.

According to one embodiment of the present disclosure, the grafted ethylenic polymer is a grafted acrylic polymer.

The grafted ethylenic polymer may be, for example, obtained by free-radical polymerization in an organic polymerization medium:
of at least one ethylenic monomer, such as of at least one acrylic monomer and optionally of at least one additional non-acrylic vinyl monomer, to form the said insoluble skeleton; and
of at least one macromonomer comprising a polymerizable end group to form the side chains, the said macromonomer having a weight-average molecular mass of greater than or equal to 200 and the polymerized macromonomer being present in an amount ranging from 0.05% to 20% by weight, relative to the weight of the polymer.

The liquid fatty phase may comprise the organic polymerization medium for the grafted ethylenic polymer.

The organic liquid dispersion medium, corresponding to the medium in which the grafted polymer is supplied, may be identical to the polymerization medium.

However, the polymerization medium may be totally or partially replaced with another organic liquid medium. This other organic liquid medium may be added, after polymerization, to the polymerization medium. The polymerization medium is then totally or partially evaporated.

The liquid fatty phase may comprise liquid organic compounds other than those present in the dispersion medium. These other compounds are chosen such that the grafted polymer remains in dispersed form in the liquid fatty phase.

The organic liquid dispersion medium is present in the liquid fatty phase of the composition according to the present disclosure due to the introduction into the composition of the dispersion of the grafted polymer obtained.

The liquid fatty phase may be a non-silicone liquid fatty phase.

As used herein, the term “non-silicone liquid fatty phase” is understood to mean a fatty phase comprising at least one non-silicone liquid organic compounds or oils, such as those mentioned above, wherein the non-silicone compounds are predominantly present in the liquid fatty phase, that is, present in an amount greater than or equal to 50% by weight, for instance from 50% to 100% by weight, such as from 60% to 100% by weight (for example from 60% to 99% by weight), or alternatively from 65% to 100% by weight (for example from 65% to 95% by weight), relative to the total weight of the liquid fatty phase.

The non-silicone liquid organic compounds may be chosen from, by way of non-limiting example:
non-silicone liquid organic compounds with a global solubility parameter according to the Hansen solubility space of less than or equal to 18 (MPa)^{1/2},
monoalcohols with a global solubility parameter according to the Hansen solubility space of less than or equal to 20 (MPa)^{1/2}; and
mixtures thereof.

The non-silicone liquid fatty phase may thus optionally comprise silicone liquid organic compounds or oils, such as those mentioned previously, which may be present in an amount less than 50% by weight, for instance ranging from 0.1% to 40% by weight, such as ranging from 1% to 35% by weight, or alternatively ranging from 5% to 30% by weight, relative to the total weight of the liquid fatty phase.

According to one embodiment of the present disclosure, the non-silicone liquid fatty phase does not comprise any silicone liquid organic compounds or oils.

When the liquid fatty phase is a non-silicone liquid fatty phase, the macromonomers present in the grafted polymer can be for example, carbon-based macromonomers as described below.

For instance, when the liquid fatty phase is a non-silicone liquid fatty phase, the grafted polymer present in the composition can be, for example, a non-silicone grafted polymer.

As used herein, the term “non-silicone grafted polymer” is understood to mean a grafted polymer predominantly comprising a carbon-based macromonomer and optionally comprising a silicone macromonomer in an amount less than or equal to 7% by weight, such as less than or equal to 5% by weight, or even being free of silicone macromonomer.

The choice of monomers constituting the skeleton of the polymer, of macromonomers, the molecular weight of the polymer, and the proportion of the monomers and macromonomers may be made as a function of the liquid organic dispersion medium so as to obtain a dispersion of particles of grafted polymers, for instance a stable dispersion, this choice possibly being made by a person skilled in the art.

As used herein, the term “stable dispersion” means a dispersion that is not liable to form a solid deposit or to undergo liquid/solid phase separation, for instance after centrifugation, for example at 4000 rpm for 15 minutes.

As used herein, the term “grafted acrylic polymer” is understood to mean a polymer that may be obtained by free-radical polymerization:
of at least one acrylic monomer and optionally of at least one additional non-acrylic vinyl monomer;
with at least one macromonomer, in an organic polymerization medium.

For example, the acrylic monomers can be present in an amount ranging from 50% to 100% by weight, for instance from 55% to 100% by weight (such as from 55% to 95% by weight) and for example from 60% to 100% by weight (such as from 60% to 90% by weight) of the mixture of acrylic monomers with optional non-acrylic vinyl monomers.

For instance, the acrylic monomers can be chosen from monomers whose homopolymer is insoluble in the dispersion medium under consideration, i.e., the homopolymer is in solid (or non-dissolved) form at a concentration of greater than or equal to 5% by weight at room temperature (20° C.) in the dispersion medium.

a) Macromonomers:

As used herein, the expression “macromonomer comprising a polymerizable end group” is understood to mean any polymer comprising on only one of its ends a polymerizable end group capable of reacting during the polymerization reaction with acrylic monomers and optionally the additional non-acrylic vinyl monomers comprising the skeleton. The macromonomer can makes it possible to form the side chains of the grafted acrylic polymer. The polymerizable group of the macromonomer may be, for example, an ethylenically unsaturated group capable of free-radical polymerization with the monomers comprising the skeleton.

As used herein, the term “carbon-based macromonomer” is understood to mean a non-silicone-based macromonomer, for instance, an oligomeric macromonomer obtained by polymerization of ethylenically unsaturated non-silicone monomer(s), and such as by polymerization of acrylic and/or non-acrylic vinyl monomers.

For example, the macromonomer can be chosen from macromonomers whose homopolymer is soluble in the dispersion medium under consideration, i.e. fully dissolved in the dispersion medium at a concentration of greater than or equal to 5% by weight and at room temperature.

Thus, the grafted acrylic polymer comprises a skeleton (or main chain) comprising a sequence of acrylic units resulting from the polymerization for example, of at least one acrylic monomers and of side chains (or grafts) derived from the reaction of the macromonomers, the side chains being covalently bonded to the said main chain.

The skeleton (or main chain) is insoluble in the dispersion medium under consideration, whereas the side chains (or grafts) are soluble in the said dispersion medium.

b) Monomers:

As used herein, the term “acrylic monomers” is understood to mean monomers chosen from (meth)acrylic acid, (meth)acrylic acid esters (also known as (meth)acrylates), and (meth)acrylic acid amides (also known as (meth)acrylamides).

Among the acrylic monomers that may be used, non-limiting mention may be made of methyl, ethyl, propyl, butyl and isobutyl(meth)acrylates; methoxyethyl or ethoxyethyl(meth)acrylates; trifluoroethyl methacrylate; dimethylaminoethyl methacrylate, diethylaminoethyl methacrylate, 2-hydroxypropyl methacrylate, 2-hydroxyethyl methacrylate, 2-hydroxypropyl acrylate, 2-hydroxyethyl acrylate; dimethylaminopropylmethacrylamide; and the salts thereof; and mixtures thereof.

By way of non-limiting example, the acrylic monomers can be chosen from methyl acrylate, methoxyethyl acrylate, methyl methacrylate, 2-hydroxyethyl methacrylate, acrylic acid and dimethylaminoethyl methacrylate, and mixtures thereof.

For instance, the acrylic monomers present in the grafted polymer can comprise at least (meth)acrylic acid and at least one monomer chosen from the (meth)acrylates and (meth)acrylamides described previously herein. For example, the acrylic monomers can comprise at least (meth)acrylic acid and at least one monomer chosen from C₁-C₃ alkyl (meth)acrylates. The (meth)acrylic acid may be present in an amount greater than or equal to 5%.by weight, such as ranging from 5% to 80% by weight, for instance greater than or equal to 10% by weight, such as ranging from 10% to 70% by weight, and for example, greater than or equal to 15% by weight, such as ranging from 15% to 60% by weight, relative to the total weight of the polymer.

Among the main acrylic monomers that may be used, non-limiting mention may be made of methyl acrylate, methyl methacrylate, ethyl acrylate, ethyl methacrylate, n-propyl acrylate, n-propyl methacrylate, isopropyl acrylate and isopropyl methacrylate, and. mixtures thereof.

In one embodiment of the present disclosure, methyl acrylate, methyl methacrylate and ethyl methacrylate are used.

The additional acrylic monomers may be chosen from, by way of non-limiting example:
(meth)acrylic acid and its salts,
the (meth)acrylates of formula (I), and salts thereof: 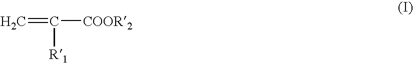
   in which:
R′₁ is chosen from a hydrogen atom and a methyl group;
R′₂ is chosen from
linear and branched alkyl groups comprising from 1 to 6 carbon atoms, the group comprising in its chain at least one oxygen atom and/or comprising at least one substituent chosen from OH groups, halogen atoms (such as F, Cl, Br and I) and —NR′R″ groups, with R′ and R″, which may be identical or different, being chosen from linear or branched C₁-C₃ alkyls;
cyclic alkyl groups comprising from 3 to 6 carbon atoms, the group possibly comprising in its chain at least one oxygen atom and/or possibly comprising at least one substituent chosen from OH groups and halogen atoms (such as F, Cl, Br or I);
and mixtures thereof.

Examples of R′₂ that may be mentioned in a non-limiting manner include methoxyethyl, ethoxyethyl, trifluoroethyl, 2-hydroxyethyl, 2-hydroxypropyl, dimethylaminoethyl, diethylaminoethyl and dimethylaminopropyl groups.

Among these additional acrylic monomers, non-limiting mention may be made for example, of (meth)acrylic acid, methoxyethyl or ethoxyethyl(meth)acrylates; trifluoroethyl methacrylate; dimethylaminoethyl methacrylate, diethylaminoethyl methacrylate, 2-hydroxypropyl methacrylate, 2-hydroxyethyl methacrylate, 2-hydroxypropyl acrylate and 2-hydroxyethyl acrylate, the salts thereof, and mixtures thereof.

In at least one embodiment, acrylic acid and methacrylic acid are used.

The macromonomers comprise, at one of the ends of the chain, a polymerizable end group capable of reacting during the polymerization with the acrylic monomers and optionally the additional vinyl monomers, to form the side chains of the grafted ethylenic polymer. The polymerizable end group can be, for instance, chosen from a vinyl or (meth)acrylate (or (meth)acryloxy) group, such as (meth)acrylate group.

The macromonomers can be chosen from, for example, macromonomers whose homopolymer has a glass transition temperature (Tg) of less than or equal to 25° C., such as ranging from −100° C. to 25° C., and for instance ranging from −80° C. to 0° C.

The macromonomers have a weight-average molecular mass of greater than or equal to 200, for example greater than or equal to 300, such as greater than or equal to 500, for instance greater than 600.

For example, the macromonomers can have a weight-average molecular mass (Mw) ranging from 200 to 100,000, for instance ranging from 500 to 50,000, such as ranging from 800 to 20,000, from 800 to 10,000 and from 800 to 6,000.

As used herein, the weight-average (Mw) and number-average (Mn) molar masses are determined by gel permeation liquid chromatography (THF solvent, calibration curve established with linear polystyrene standards, refractometric detector).

Carbon-based macromonomers that may be used include, by way of non-limiting example:
(i) homopolymers and copolymers of linear or branched C₈-C₂₂ alkyl(meth)acrylate, comprising a polymerizable end group chosen from vinyl or (meth)acrylate groups, among which non-limiting mention may be made, for example, of: poly(2-ethylhexyl acrylate) macromonomers with a mono(meth)acrylate end group; poly(dodecyl acrylate) or poly(dodecyl methacrylate) macromonomers with a mono(meth)acrylate end group; poly(stearyl acrylate) or poly(stearyl methacrylate) macromonomers with a mono(meth)acrylate end group. Such macromonomers are described for instance in European Patent Nos. EP 0 895 467 and EP 0 096 459, and in the article by Gillman K. F., Polymer Letters, Vol 5, page 477-481 (1967). Non-limiting mention may be made, for instance, of macromonomers based on poly(2-ethylhexyl acrylate) or poly(dodecyl acrylate) with a mono(meth)acrylate end group;
(ii) polyolefins comprising an ethylenically unsaturated end group, for instance comprising a (meth)acrylate end group. Non-limiting examples of such polyolefins that may be mentioned include, for instance, the following macromonomers, it being understood that they have a (meth)acrylate end group: polyethylene macromonomers, polypropylene macromonomers, macromonomers of polyethylene/polypropylene copolymer, macromonomers of polyethylene/polybutylene copolymer, polyisobutylene macromonomers; polybutadiene macromonomers; polyisoprene macromonomers; polybutadiene macromonomers; poly(ethylene/butylene)-polyisoprene macromonomers.

Such macromonomers are described for example in U.S. Pat. No. 5,625,005, which mentions ethylene/butylene and ethylene/propylene macromonomers comprising a (meth)acrylate reactive end group.

Further non-limiting mention may be made of, for example, poly(ethylene/butylene)methacrylate such as the product sold under the name Kraton Liquid L-1253 by Kraton Polymers.

For example, the grafted polymer can have a weight-average molecular mass (Mw) ranging from 10,000 to 300,000, for instance ranging from 20,000 to 200,000, such as from 25,000 to 150,000.

The dispersion of grafted polymer particles can have, for example, a polymer solids content (or dry extract) ranging from 40% to 70% by weight of solids, such as ranging from 45% to 65% by weight of solids.

The grafted polymer may be present in the at least one first composition according to the present disclosure in a solids content (or active material content) ranging from 1% to 70% by weight, for instance from 5% to 60% by weight, such as ranging from 6% to 45% by weight and from 8% to 40% by weight, relative to the total weight of the composition.

The grafted ethylenic polymer is described in greater detail in the French Patent Application Publication No. FR 2 863 493 (of French Patent Application No. FR 04/50540), the content of which is incorporated herein by reference.

Plasticizer

In the context of the present disclosure, at least one plasticizer for the particular polymer may be combined with the at least one film-forming polymer, so as to lower the Tg of the polymer film and to improve the adhesion of the polymer film to its support, in particular keratin materials. The at least one plasticizer can lower the glass transition temperature of the polymer by at least 1, 2, 3 or 4° C., and for instance from 5° C. to 20° C. In one embodiment, for example, the at least one plasticizer lowers the glass, transition temperature of the polymer by at least 1, 2, 3 or 4° C., such as from 5° C. to 20° C., when the at least one plasticizer is present in an amount less than 10% by weight of the polymer.

According to another embodiment, the at least one plasticizer for the polymer is chosen from plasticizers with a solubility parameter δₕ ranging from 5.5 (J/cm³)^{1/2} to 11 (J/cm³)^{1/2,} for instance from 5.9 (J/cm³)^{1/2} to 11 (J/cm³)^{1/2}, such as from 7 (J/cm³)^{1/2} to 10.5 (J/cm³)^{1/2}, and from 9 (J/cm³)^{1/2}to 10 (J/cm³)^{1/2}. In one embodiment it ranges from 8 (J/cm³)^{1/2} to 10 (J/cm³)^{1/2}.

The solubility parameter δₚ of the at least one plasticizer can range, for example, from 1.5 (J/cm³)^{1/2} to 4.5 (J/cm³)^{1/2}, for instance from 1.5 (J/cm³)^{1/2} to 4 (J/cm³)^{1/2}, such as from 1.5 (J/cm³)^{1/2}t o 3.5 (J/cm³)^{1/2} and from 2 (J/cm³)^{1/2} and 3 (J/cm³)^{1/2}.

The definition of the solubility parameters according to Hansen is well known to those skilled in the art and is described for instance, in the article by C. M. Hansen: “The three dimensional solubility parameters” J. Paint Technol. 39, 105 (1967). These parameters are also described in Japanese Patent No. JP-A-08-109 121 from KAO and the publication from D. W. Van Krevelen “Properties of polymers” (1990), p. 190.

It is within the capacity of a person skilled in the art to determine the amounts of each plasticizer that may be used in order to obtain a mixture of plasticizers that satisfies the above relationships.

In one embodiment, the at least one plasticizer may be chosen from esters of at least one carboxylic acid comprising from 1 to 7 carbon atoms and of a polyol comprising at least four hydroxyl groups.

The polyol may be a monosaccharide or a polysaccharide comprising 1 to 10 saccharides, such as from 1 to 4 saccharides, and for instance, one or two saccharides. The polyol may be chosen from erythritol, xylitol, sorbitol, glucose and sucrose.

The ester may be obtained by copolymerization of two esters according to the present disclosure, for instance by copolymerization (i) of a sucrose substituted with benzoyl groups and (ii) of a sucrose substituted with acetyl and/or isobutyryl groups.

The ester may also be obtained from at least two different monocarboxylic acids. According to one embodiment, the acid is an unsubstituted linear or branched acid.

The acid can be, for example, a monocarboxylic acid chosen for instance from acids comprising from 1 to 7 carbon atoms, such as from 1 to 5 carbon atoms, for example acetic acid, n-propanoic acid, isopropanoic acid, n-butanoic acid, isobutanoic acid, tert-butanoic acid, n-pentanoic acid and benzoic acid. In one embodiment, the acid is can be chosen from acetic acid, isobutyric acid and benzoic acid.

According to another embodiment, the ester is sucrose diacetate hexakis(2-methylpropanoate).

According to still another embodiment, the at least one plasticizer according to the present disclosure is chosen from esters of aliphatic or aromatic polycarboxylic acids and of aliphatic or aromatic alcohols comprising from 1 to 10 carbon atoms.

Among the citrates that may be used, non-limiting mention may be made of tributyl acetyl citrate, triethyl acetyl citrate, triethylhexyl acetyl citrate, trihexyl acetyl citrate, trihexyl butyryl citrate, isodecyl citrate, isopropyl citrate, tributyl citrate and triethylhexyl citrate. According to one embodiment, the carboxylic acid is not tributyl acetyl citrate.

Among the adipates that may be used, non-limiting mention may be made of dibutyl adipate and 2-diethylhexyl adipate. According to another embodiment, the plasticizer according to the present disclosure is not diisopropyl adipate.

Among the sebacates that may be used, non-limiting mention may be made of dibutyl sebacate, diethylhexyl sebacate, diethyl sebacate and diisopropyl sebacate.

Among the succinates that may be used, non-limiting mention may be made of diethylhexyl succinate and diethyl succinate.

The at least one plasticizer for example, does not have to comprise any polar groups, such as any hydroxyl groups. The “polar groups” are, for example, ionic or nonionic polar groups chosen from —COOH; —OH; ethylene oxide; propylene oxide; —PO₄; —NHR; and —NR₁R₂ groups, wherein R₁ and R₂ optionally form a ring, and are chosen from linear and branched C₁ to C₂₀ alkyl and alkoxy radicals.

The at least one plasticizer may be present in an amount ranging from 0.1% to 25% by weight, for instance from 0.5% to 15% by weight, such as from 3% to 15% by weight.

The mass ratio.between the at least one film-forming polymer and the at least one plasticizer can range, for example, from 0.5 to 100, for instance, from 1 to 50, such as from 1 to 10 and from 1 to 5.

The at least one first composition can be, for example, one of the compositions described in the French Patent Application Publication No. FR 2 851 467 (of French Patent Application No. FR 03/50034), the content of which is incorporated herein by reference.

Wax

The at least one first composition of the product according to the present disclosure may furthermore comprise at least one wax.

As used herein, the term “wax” is understood to mean a lipophilic fatty compound, which is solid at room temperature (25° C.), with a reversible solid/liquid change of state, which has a melting point of greater than 40° C. that may be up to 200° C., and which has in the solid state an anisotropic crystal organization.

For example, non-limiting mention may be made of linear hydrocarbon-based waxes, which may be used according to the context of the present disclosure. Their melting point can be, for instance greater than or equal to 35° C., for example greater than or equal to 55° C., such as greater than or equal to 80° C.

The linear hydrocarbon-based waxes can be chosen from, by way of non-limiting example, substituted linear alkanes, unsubstituted linear alkanes, unsubstituted linear alkenes and substituted linear alkenes, and an unsubstituted compound being exclusively composed of carbon and hydrogen. The substituents mentioned above do not comprise any carbon atoms.

The linear hydrocarbon-based waxes include ethylene polymers and copolymers with a molecular weight ranging from 400 to 800, for example Polywax 500 or Polywax 400 sold by New Phase Technologies.

The linear hydrocarbon-based waxes include linear paraffin waxes, for instance the paraffins S&P 206, S&P 173 and S&P 434 from Strahl & Pitsch.

The linear hydrocarbon-based waxes further include long-chain linear alcohols, for instance products comprising a mixture of polyethylene and of alcohols comprising from 20 to 50 carbon atoms, such as Performacol 425 or Performacol 550 (mixture in 20/80 proportions) sold by New Phase Technologies.

The at least one waxe may be present in an amount ranging from 2% to 30% by weight, for instance from 5% to 20%, and such as from 5% to 15% by weight, relative to the weight of the at least one first, so as not to excessively reduce the gloss of the composition and of the film deposited on the lips and/or the skin.

Coloring Agent

The at least one first and/or the at least one second composition of the cosmetic product according to the present disclosure may comprise at least one coloring agent that can be chosen from water-soluble dyes, liposoluble dyes, pigments, and nacres.

As used herein, the term “pigments” is understood to mean white or colored, mineral or organic particles, which are insoluble in the liquid organic phase and which are intended to color and/or opacify the composition.

As used herein, the term “nacres” is understood to mean iridescent particles produced for example, by certain molluscs in their shell, or else synthesized, which are insoluble in the medium of the at least one first composition.

As used herein, the term “dyes” is understood to mean generally organic compounds that are soluble in fatty substances, for instance oils, or soluble in an aqueous-alcoholic phase.

The liposoluble dyes can be, by way of non-limiting example, Sudan Red, D&C Red No. 17, D&C Green No. 6, β-carotene, soybean oil, Sudan Brown, D&C Yellow No. 11, D&C Violet No. 2, D&C Orange No. 5, quinoline yellow, annatto and bromo acids.

The water-soluble dyes can be, by way of non-limiting example, beetroot juice, methylene blue and caramel.

The pigments may be interference or non-interference, mineral and/or organic, white or colored pigments. Among the mineral pigments that may be mentioned in a non-limiting manner include titanium dioxide, optionally surface-treated, zirconium oxide or cerium oxide, and also zinc oxide, iron oxide (black, yellow, brown or red) or chromium oxide, manganese violet, ultramarine blue, chromium hydrate and ferric blue. Among the organic pigments that may be mentioned in a non-limiting manner include carbon black, pigments of barium, strontium, calcium or aluminium organic lake type, including those submitted for certification by:the Food and Drug Administration (FDA) (for example D&C or FD&C) and those exempt from FDA certification, for instance lakes based on cochineal carmine.

The nacres or nacreous pigments may be chosen from white nacreous pigments such as mica coated with titanium or with bismuth oxychloride, colored nacreous pigments such as titanium mica with iron oxides, titanium mica with, for instance, ferric blue or chromium oxide, titanium mica with an organic pigment of the abovementioned type, and also nacreous pigments based on bismuth oxychloride. Pigments with goniochromatic properties and/or pigments with a metallic effect as described in the French Patent Application Publication No. FR 2 842 417 (of French Patent Application No. FR 0209246), the content of which is incorporated herein by reference.

The at least one coloring agent, when present, can be present in an amount ranging from 0.001% to 60% by weight, for instance from 0.01% to 50% by weight, such as from 0.1% to 40% by weight, relative to the total weight of the at least one first or at least one second composition.

Fillers

The at least one first and/or at least one second composition according to the present disclosure may comprise at least one filler, for example in an amount ranging from 0.01% to 50% by weight, such as ranging from 0.01% to 30% by weight, relative to the total weight of the composition comprising the at least one filler. As used herein, the term “fillers” is understood to mean colorless or white, mineral or synthetic particles of any form, which are insoluble in the medium of the composition irrespective of the temperature at which the composition is manufactured. These fillers can serve, for instance, to modify the rheology or the texture of the composition.

The fillers may be mineral or organic, of any form, platelet-shaped, spherical or oblong, irrespective of the crystallographic form (for example lamellar, cubic, hexagonal, orthorhombic, etc.). Non-limiting mention may be made of talc, mica, silica, kaolin, polyamide (Nylon®) (Orgasol® from Atochem) powder, poly-β-alanine powder and polyethylene powder, powders of tetrafluoroethylene polymers (Teflon®), lauroyllysine, starch, boron nitride, hollow polymer microspheres such as those of polyvinylidene chloride/acrylonitrile, for instance Expancel® (Nobel Industrie), or of acrylic acid copolymers (Polytrap® from the company Dow Corning) and silicone resin microbeads (for example Tospearls® from Toshiba), elastomeric polyorganosiloxane particles, precipitated calcium carbonate, magnesium carbonate, magnesium hydrocarbonate, hydroxyapatite, hollow silica microspheres (Silica Beads® from Maprecos), glass or ceramic microcapsules, and metal soaps derived from organic carboxylic acids comprising from 8 to 22 carbon atoms, such as from 12 to 18 carbon atoms, for example zinc stearate, magnesium stearate, lithium stearate, zinc laurate or magnesium myristate.

The at least one second composition can comprise, for example, less than 15%, for instance less than 10%, such as less than 5% by weight of at least one coloring agent and/or at least one filler.

Adjuvants

The at least one first and/or at least one second composition may also comprise at least one adjuvant usually used in such compositions, such as active agents, water, antioxidants, fragrances, surfactants, preserving agents and essential oils.

Among the cosmetic, dermatological, hygiene or pharmaceutical active agents that may be used in the at least one first and/or at least one second compositions of the present disclosure, non-limiting mention may be made of moisturizers, vitamins, essential fatty acids, sphingolipids and sunscreens. These active agents are used in an amount that is usual for a person skilled in the art, for example, in an amount ranging from 0% to 20% by weight, such as from 0.001% to 15% by weight, relative to the total weight of the composition comprising the at least one adjuvant.

Needless to say, a person skilled in the art will take care to select this or these optional additional compound(s), and/or the amount thereof, such that the beneficial properties of the compositions and/or product according to the present disclosure are not, or are not substantially, adversely affected by the envisaged addition.

Galenical Forms

A lip makeup product according to the present disclosure can comprise, for example, at least one first composition in the form of a stick or in liquid form, the at least one second composition being, for example, in fluid form packaged in a tube. The product according to the present disclosure comprises at least two physiologically acceptable compositions that can be packaged separately or together in the same packaging article or in at least two separate or different packaging articles.

In one embodiment of the present disclosure, the at least one first and at least one second compositions are preferably packaged separately.

The present disclosure also relates to a makeup kit comprising a cosmetic makeup product as defined above, in which the at least two compositions are separately packaged and are accompanied by suitable application devices, or can be applied by finger. These devices may be fine brushes, coarse brushes, pens, pencils, felts, quills, sponges, tubes and/or foam tips. According to one embodiment, the at least one first composition is fluid and applied using a foam tip, while the at least one second composition is fluid and applied using a tube. According to another embodiment, the at least one first composition is a stick, while the at least one second composition is fluid and is applied using a tube. According to still another embodiment, the at least one first composition is fluid and the at least one second composition is in the form of a stick. According to yet another embodiment, the at least two compositions are in the form of a stick.

The present disclosure also relates to a process for making up the skin and/or the lips and/or the integuments, which comprises applying to the skin, the lips and/or the integuments at least one coat of the at least one first composition comprising a physiologically acceptable medium, and applying, onto all or part of the at least one first coat, at least one coat of the at least one second composition.

The present disclosure also relates to a made-up support comprising at least one coat of at least one first composition comprising a physiologically acceptable medium and at least one coat of at least one second composition deposited onto all or part of the at least one first coat, wherein the at least one second composition comprises at least one silicone polymer with a viscosity, measured according to standard ASTM D-445, ranging from 10 cSt to 10,000 cSt.

In one embodiment of the present disclosure, for example, the compositions according to the present disclosure may be prepared in the usual manner for those skilled in the art. They may be in the form of a cast product, for example in the form of a stick or wand, or in the form of a dish that may be used by direct contact or with a sponge. For example, they can find an application as cast foundations, cast makeup rouges, cast eyeshadows, lipsticks, lipcare bases or balms and concealer products. They may also be in the form of a soft paste or alternatively a gel, a more or less fluid cream, or a liquid packaged in a tube. They may then be in the form of foundations or lipsticks, antisun products or skin coloring products.

These compositions for topical application may be, for example, a cosmetic, dermatological, hygiene or pharmaceutical composition for protecting, treating or caring for the face, the neck, the hands or the body (for example care cream, antisun oil or body gel), a makeup composition (for example a makeup gel, cream or stick) or an artificial tanning or skin protecting composition.

The at least one first and/or at least one second composition according to the present disclosure may be in the form of a dermatological or care composition for the skin and/or the integuments or in the form of an antisun composition or a body hygiene composition, such as in the form of a deodorant. In one embodiment of the present disclosure, they are in uncolored form. They may then be used as a care base for the skin, the integuments or the lips (lip balms, for protecting the lips against the cold and/or sunlight and/or the wind, or care creams for the skin, the nails or the hair).

The at least one first and/or at least one second composition according to the present disclosure, in one embodiment, are lipsticks in the form of a stick or in fluid form.

Each composition of the at least two-coat makeup product according to the present disclosure can be in any galenical form normally used for topical application, for instance in the form of an oily or aqueous solution, an oily or aqueous gel, an oil-in-water or water-in-oil emulsion, a multiple emulsion, a dispersion of oil in water using vesicles, the vesicles being located at the oil/water interface, or a powder. Each composition may be fluid or solid.

For example, the at least one first and/or the at least one second composition can have a continuous fatty phase, such as in anhydrous form, and may comprise less than 5% water, for instance less than 1% water by weight, relative to the total weight of the at least one first or at least one second composition. For example, in one embodiment of the present disclosure, the makeup product is in anhydrous form.

Each composition may be packaged separately in the same packaging article, for example in a pen with at least two compartments, the at least one first composition being delivered from one end of the pen and the at least one second composition being delivered from the other end of the pen, each end being closed, for instance, in a leaktight manner with a cap.

For example, in one embodiment, the at least one first composition that is applied as the at least one first coat is in solid form, which can allow a more practical application, better stability of the composition over time, and with respect to temperature, and can allow precise application of the makeup, which can be highly desirable in the case of a lipstick or an eyeliner.

In one embodiment, the product is a lipstick.

In another embodiment, the at least one first and/or at least one second composition is in solid form.

In still another embodiment, the at least one top coat has care, gloss and transparency properties.

The present disclosure also relates to a lip product, a foundation, a tattoo, a makeup rouge or an eyeshadow comprising at least one first and at least one second composition as described above.

The at least one first and at least one second compositions of the present disclosure may be obtained by heating the various constituents to the melting point of the highest-melting waxes, followed by casting the molten mixture in a mould (dish or finger stall). They may also be obtained by extrusion as described in European Patent Application No. EP-A-0 667 146.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements.

The following examples illustrate the present disclosure in a non-limiting manner. The percentages are weight percentages.

### EXAMPLES

### Example

### Lipstick Product

First composition

| Constituent | wt % |
|---|---|
| Polyethylene wax | 10.5% |
| (weight-average molecular mass 500) | |
| Linear fatty alcohols | 2.5% |
| (Performacol 550 Alcohol sold by New Phase Technologies) | |
| Dispersion of Example 1 of European Patent Application | 68% |
| Publication No. EP-A-749 746 | |
| Sucrose acetate isobutyrate | 5% |
| (Eastman SAIB sold by Eastman Chemical) | |
| Pigmentary paste | 13.5% |
| Fragrance | 0.5% |

A dispersion of non-crosslinked copolymer of methyl acrylate and of acrylic acid in a 95/5 ratio, in isododecane, was prepared according to the method of Example 1 of European Patent Application Publication No. EP-A-749 746, replacing the heptane with isododecane. A dispersion of poly(methyl acrylate/acrylic acid) particles surface-stabilized in isododecane with a polystyrene/copoly(ethylene-propylene)diblock copolymer sold under the name Kraton G1701, having a solids content of 25% by weight, was thus obtained.

The polyethylene wax, the C₃₀₋₅₀ alcohols and the pigmentary paste were introduced into a heating pan and heated to 100° C. with magnetic stirring so as to obtain a homogeneous mixture. The pigmentary paste comprised 70% pigments,1% poly(12-hydroxystearic acid)stearate and 29% hydrogenated polyisobutene.

The dispersion according to Example 1 of European Patent Application Publication No. EP-A-749 746, and the sucrose acetate isobutyrate were then added while maintaining the temperature and the stirring until the mixture was homogeneous. The composition was poured into molds. The sticks obtained were of homogeneous shade and were slippery on application. They gave a deposit on the lips that had good staying power, did not migrate, did not transfer and was not tacky.

Second composition

| Constituent | Wt % |
|---|---|
| Polydimethylsiloxane sold under the reference AK 300000 by | 33% |
| Wacker (300,000 cSt) | |
| Polydimethylsiloxane sold under the reference DC 200 (350 cSt) | 43% |
| by Dow Corning | |
| Mixture of α,ω-dihydroxylated polydimethylsiloxane and of | 14% |
| polydimethylsiloxane 5 cSt sold under the reference DC 2-9085 by | |
| Dow Corning | |
| Cyclopentasiloxane | 10% |

The staying power of the deposit of the makeup product was evaluated according to the protocol described above. It was equal to 69%.

Another example of a second composition

| Constituent | Wt % |
|---|---|
| Polydimethylsiloxane sold under the reference | 50% |
| Dow Corning 200 Fluid 60000 cSt | |
| Polydimethylsiloxane sold under the reference | 43% |
| Dow Corning 200 Fluid 350 cSt | |
| Cyclohexadimethylsiloxane | 7% |
| Dow Corning 246 Fluid | |

The staying power of the deposit of the makeup product was evaluated according to the protocol described above. It was equal to 73%.

What is claimed is:

1. A cosmetic product comprising at least one first composition and at least one second composition, wherein
the at least one first composition comprises a physiologically acceptable medium and, optionally, at least one acrylic polymer, and
the at least one second composition comprises at least one silicone polymer with a viscosity at 25° C., measured according to standard ASTM D-445, ranging from 5 cSt to 10,000 cSt, the at least one silicone polymer being present in a sufficient amount in the at least one second composition such that the ratio (i)/(ii) between (i) the staying power of a deposit of the product resulting from the successive application of the at least one first and the at least one second compositions and (ii) the staying power of a deposit of the at least one first composition is greater than 1.1,
and wherein the staying power of a deposit of the at least one first composition is greater than or equal to 30%.

2. A cosmetic product comprising at least one first composition and at least one second composition, wherein
the at least one first composition comprises a physiologically acceptable medium and, optionally, at least one acrylic polymer, and
the at least one second composition comprises at least one first silicone polymer chosen from polydimethylsiloxanes with a viscosity at 25° C., measured according to standard ASTM D-445, ranging from 5 cSt to 10,000 cSt, and at least one second silicone polymer chosen from polydimethylsiloxanes with a viscosity at 25° C., measured according to standard ASTM D-445, ranging from 5,000 cSt to 300,000 cSt,
and wherein the viscosity of the at least one second silicone polymer is greater than the viscosity of the at least one first silicone polymer.

3. A cosmetic product comprising at least one first composition and at least one second composition, wherein
the at least one first composition comprises a physiologically acceptable medium and, optionally, at least one acrylic polymer, and
the at least one second composition comprises at least one first silicone polymer with a viscosity at 25° C., measured according to standard ASTM D-445, ranging from 50 cSt to 1,000 cSt, and at least one second silicone polymer with a viscosity at 25° C., measured according to standard ASTM D-445, ranging from 30,000 cSt to 300,000 cSt.

4. A cosmetic product comprising at least one first composition and at least one second composition, wherein
the at least one first composition comprises a physiologically acceptable medium and, optionally, at least one acrylic polymer, and
the at least one second composition comprises at least one silicone polymer with a viscosity at 25° C., measured according to standard ASTM D-445, ranging from 5 cSt to 10,000 cSt, the at least one silicone polymer being present in a sufficient amount in the at least one second composition such that the staying power of a deposit of the product resulting from the application of the at least one first and at least one second compositions is greater than 65%.

5. The cosmetic product according to claim 1, wherein the ratio (i)/(ii) between (i) the staying power of-a deposit of the product and (ii) the staying power of the at least one first composition is greater than or equal to 1.20.

6. The cosmetic product according to claim 1, wherein the staying power of a deposit of the at least one first composition is greater than or equal to 55%.

7. The cosmetic product according to claim 5, wherein the staying power of a deposit of the at least one first composition is greater than or equal to 55%.

8. The cosmetic product according to claim 1, wherein the at least one silicone polymer has a viscosity ranging from 100 cSt to 500 cSt.

9. The cosmetic product according to claim 2, wherein the at least one first silicone polymer has a viscosity ranging from 100 cSt to 500 cSt.

10. The cosmetic product according to claim 4, wherein the at least one silicone polymer has a viscosity ranging from 100 cSt to 500 cSt.

11. The cosmetic product according to claim 1, wherein the at least one second composition has a viscosity at 25° C. ranging from 3 Pa·s. to 25 Pa·s.

12. The cosmetic product according to claim 2, wherein the at least one second composition has a viscosity at 25° C. ranging from 3 Pa·s. to 25 Pa·s.

13. The cosmetic product according to claim 3, wherein the at least one second composition has a viscosity at 25° C. ranging from 3 Pa·s. to 25 Pa·s.

14. The cosmetic product according to claim 4, wherein the at least one second composition has a viscosity at 25° C. ranging from 3 Pa·s. to 25 Pa·s.

15. The cosmetic product according to claim 1, wherein the at least one second composition comprises only one silicone polymer and wherein this silicone polymer has a viscosity at 25° C. ranging from 3 Pa·s to 6 Pa·s.

16. The cosmetic product according to claim 4, wherein the at least one second composition comprises only one silicone polymer and wherein this silicone polymer has a viscosity at 250C ranging from 3 Pa·s to 6 Pa·s.

17. The cosmetic product according to claim 1, wherein the at least one second composition comprises at least one second silicone polymer with a viscosity ranging from 5,000 cSt to 300,000 cSt, and wherein the viscosity of the at least one second silicone is greater than the viscosity of the at least one first silicone polymer.

18. The cosmetic product according to claim 4, wherein the at least one second composition comprises at least one second silicone polymer with a viscosity ranging from 5,000 cSt to 300,000 cSt, and wherein the viscosity of the at least one second silicone is greater than the viscosity of the at least one first silicone polymer.

19. The cosmetic product according to claim 2, wherein the at least one second silicone polymer has a viscosity ranging from 30,000 cSt to 100,000 cSt.

20. The cosmetic product according to claim 3, wherein the at least one second silicone polymer has a viscosity ranging from 30,000 cSt to 1 00,000 cSt.

21. The cosmetic product according to claim 2, wherein the at least one first and the at least one second silicone polymers are present in an amount such that the dynamic viscosity of the at least one second composition at 25° C., measured with a Brookfield RV rotary viscometer, ranges from 5 Pa·s to 25 Pa·s.

22. The cosmetic product according to claim 3, wherein the at least one first and the at least one second silicone polymers are present in an amount such that the dynamic viscosity of the at least one second composition at 25° C., measured with a Brookfield RV rotary viscometer, ranges from 5 Pa·s to 25 Pa·s.

23. The cosmetic product according to claim 4, wherein the staying power of the product is greater than or equal to 75%.

24. The cosmetic product according to claim 1, wherein the at least one silicone polymer is chosen from polydimethylsiloxanes.

25. The cosmetic product according to claim 3, wherein the at least one first silicone polymer is chosen from polydimethylsiloxanes.

26. The cosmetic product according to claim 4, wherein the at least one silicone polymer is chosen from polydimethylsiloxanes.

27. The cosmetic product according to claim 17, wherein the at least one second silicone polymer is chosen from polydimethylsiloxanes.

28. The cosmetic product according to claim 18, wherein the at least one second silicone polymer is chosen from polydimethylsiloxanes.

29. The cosmetic product according to claim 1, wherein the at least one second composition is transparent.

30. The cosmetic product according to claim 2, wherein the at least one second composition is transparent.

31. The cosmetic product according to claim 3, wherein the at least one second composition is transparent.

32. The cosmetic product according to claim 4, wherein the at least one second composition is transparent.

33. The cosmetic product according to claim 1, wherein the at least one second composition further comprises at least one coloring agent.

34. The cosmetic product according to claim 2, wherein the at least one second composition further comprises at least one coloring agent.

35. The cosmetic product according to claim 3, wherein the at least one second composition further comprises at least one coloring agent.

36. The cosmetic product according to claim 4, wherein the at least one second composition further comprises at least one coloring agent.

37. The cosmetic product according to claim 1, wherein the at least one second composition does not comprise wax.

38. The cosmetic product according to claim 2, wherein the at least one second composition does not comprise wax.

39. The cosmetic product according to claim 3, wherein the at least one second composition does not comprise wax.

40. The cosmetic product according to claim 4, wherein the at least one second composition does not comprise wax.

41. The cosmetic product according to claim 1, wherein the at least one second composition is liquid.

42. The cosmetic product according to claim 2, wherein the at least one second composition is liquid.

43. The cosmetic product according to claim 3, wherein the at least one second composition is liquid.

44. The cosmetic product according to claim 4, wherein the at least one second composition is liquid.

45. The cosmetic product according to claim 1, wherein the at least one second composition comprises less than 10% by weight of pigments and/or fillers.

46. The cosmetic product according to claim 2, wherein the at least one second composition comprises less than 10% by weight of pigments and/or fillers.

47. The cosmetic product according to claim 3, wherein the at least one second composition comprises less than 10% by weight of pigments and/or fillers.

48. The cosmetic product according to claim 4, wherein the at least one second composition comprises less than 10% by weight of pigments and/or fillers.

49. The cosmetic product according to claim 1, wherein the at least one second composition further comprises, in an amount less than or equal to 50% by weight, relative to the total weight of the at least one second composition, at least one silicone oil with a viscosity at 25° C., measured according to standard ASTM D-445, of less than 50 cSt.

50. The cosmetic product according to claim 2, wherein the at least one second composition further comprises, in an amount less than or equal to 50% by weight, relative to the total weight of the at least one second composition, at least one silicone oil with a viscosity at 25° C., measured according to standard ASTM D-445, of less than 50 cSt.

51. The cosmetic product according to claim 3, wherein the at least one second composition further comprises, in an amount less than or equal to 50% by weight, relative to the total weight of the at least one second composition, at least one silicone oil with a viscosity at 25° C., measured according to standard ASTM D-445, of less than 50 cSt.

52. The cosmetic product according to claim 4, wherein the at least one second composition further comprises, in an amount less than or equal to 50% by weight, relative to the total weight of the at least one second composition, at least one silicone oil with a viscosity at 25° C., measured according to standard ASTM D-445, of less than 50 cSt.

53. The cosmetic product according to claim 1, wherein the at least one first composition is solid and comprises at least one acrylic polymer.

54. The cosmetic product according to claim 2, wherein the at least one first composition is solid and comprises at least one acrylic polymer.

55. The cosmetic product according to claim 3, wherein the at least one first composition is solid and comprises at least one acrylic polymer.

56. The cosmetic product according to claim 4, wherein the at least one first composition is solid and comprises at least one acrylic polymer.

57. The cosmetic product according to claim 53, wherein the at least one first composition comprises a liquid fatty phase and at least one acrylic polymer in the form of particles dispersed in the liquid fatty phase.

58. The cosmetic product according to claim 54, wherein the at least one first composition comprises a liquid fatty phase and at least one acrylic polymer in the form of particles dispersed in the liquid fatty phase.

59. The cosmetic product according to claim 55, wherein the at least one first composition comprises a liquid fatty phase and at least one acrylic polymer in the form of particles dispersed in the liquid fatty phase.

60. The cosmetic product according to claim 56, wherein the at least one first composition comprises a liquid fatty phase and at least one acrylic polymer in the form of particles dispersed in the liquid fatty phase.

61. The cosmetic product according to claim 53, wherein the at least one acrylic polymer is present in an amount ranging from 2% to 40% by weight, relative to the total weight of the first composition.

62. The cosmetic product according to claim 54, wherein the at least one acrylic polymer is present in an amount ranging from 2% to 40% by weight, relative to the total weight of the first composition.

63. The cosmetic product according to claim 55, wherein the at least one acrylic polymer is present in an amount ranging from 2% to 40% by weight, relative to the total weight of the first composition.

64. The cosmetic product according to claim 56, wherein the at least one acrylic polymer is present in an amount ranging from 2% to 40% by weight, relative to the total weight of the first composition.

65. The cosmetic product according to claim 57, wherein the at least one first composition further comprises a stabilizer.

66. The cosmetic product according to claim 58, wherein the at least one first composition further comprises a stabilizer.

67. The cosmetic product according to claim 59, wherein the at least one first composition further comprises a stabilizer.

68. The cosmetic product according to claim 60, wherein the at least one first composition further comprises a stabilizer.

69. The cosmetic product according to claim 65, wherein the at least one stabilizer is a grafted-block or block polymer, comprising at least one block resulting from the polymerization of diene and at least one block of a vinyl polymer.

70. The cosmetic product according to claim 66, wherein the at least one stabilizer is a grafted-block or block polymer, comprising at least one block resulting from the polymerization of diene and at least one block of a vinyl polymer.

71. The cosmetic product according to claim 67, wherein the at least one stabilizer is a grafted-block or block polymer, comprising at least one block resulting from the polymerization of diene and at least one block of a vinyl polymer.

72. The cosmetic product according to claim 68, wherein the at least one stabilizer is a grafted-block or block polymer, comprising at least one block resulting from the polymerization of diene and at least one block of a vinyl polymer.

73. The cosmetic product according to claim 69, wherein the at least one stabilizer is a diblock polymer.

74. The cosmetic product according to claim 70, wherein the at least one stabilizer is a diblock polymer.

75. The cosmetic product according to claim 71, wherein the at least one stabilizer is a diblock polymer.

76. The cosmetic product according to claim 72, wherein the at least one stabilizer is a diblock polymer.

77. The cosmetic product according to claim 53, wherein the at least one acrylic polymer is a grafted acrylic polymer.

78. The cosmetic product according to claim 54, wherein the at least one acrylic polymer is a grafted acrylic polymer.

79. The cosmetic product according to claim 55, wherein the at least one acrylic polymer is a grafted acrylic polymer.

80. The cosmetic product according to claim 56, wherein the at least one acrylic polymer is a grafted acrylic polymer.

81. The cosmetic product according to claim 1, wherein the at least one first composition is in anhydrous form.

82. The cosmetic product according to claim 2, wherein the at least one first composition is in anhydrous form.

83. The cosmetic product according to claim 3, wherein the at least one first composition is in anhydrous form.

84. The cosmetic product according to claim 4, wherein the at least one first composition is in anhydrous form.

85. The cosmetic product according to claim 1, wherein the at least one second composition is in anhydrous form.

86. The cosmetic product according to claim 2, wherein the at least one second composition is in anhydrous form.

87. The cosmetic product according to claim 3, wherein the at least one second composition is in anhydrous form.

88. The cosmetic product according to claim 4, wherein the at least one second composition is in anhydrous form.

89. The cosmetic product according to claim 1, wherein it is in the form chosen from a foundation, a makeup rouge, an eyeshadow, a lipstick, a product with care properties, an eyeliner, a concealer product, or a body makeup product.

90. The cosmetic product according to claim 2, wherein it is in the form chosen from a foundation, a makeup rouge, an eyeshadow, a lipstick, a product with care properties, an eyeliner, a concealer product, or a body makeup product.

91. The cosmetic product according to claim 3, wherein it is in the form chosen from a foundation, a makeup rouge, an eyeshadow, a lipstick, a product with care properties, an eyeliner, a concealer product, or a body makeup product.

92. The cosmetic product according to claim 4, wherein it is in the form chosen from a foundation, a makeup rouge, an eyeshadow, a lipstick, a product with care properties, an eyeliner, a concealer product, or a body makeup product.

93. The cosmetic product according to claim 89, wherein said product is in the form of a lipstick.

94. The cosmetic product according to claim 90, wherein said product is in the form of a lipstick.

95. The cosmetic product according to claim 91, wherein said product is in the form of a lipstick.

96. The cosmetic product according to claim 92, wherein said product is in the form of a lipstick.

97. The cosmetic product according to claim 53, wherein the at least one first composition is liquid.

98. The cosmetic product according to claim 54, wherein the at least one first composition is liquid.

99. The cosmetic product according to claim 55, wherein the at least one first composition is liquid.

100. The cosmetic product according to claim 56, wherein the at least one first composition is liquid.

101. A makeup kit comprising a cosmetic product, said cosmetic product comprising at least one first composition and at least one second composition, wherein
the at least one first composition comprises a physiologically acceptable medium and, optionally, at least one acrylic polymer, and
the at least one second composition comprises at least one silicone polymer with a viscosity at 25° C., measured according to standard ASTM D-445, ranging from 5 cSt to 10,000 cSt, the at least one silicone polymer being present in a sufficient amount in the at least one second composition such that the ratio (i)/(ii) between (i) the staying power of a deposit of the product resulting from the successive application of the at least one first and the at least one second compositions and (ii) the staying power of a deposit of the at least one first composition is greater than 1.1,
wherein the staying power of a deposit of the at least one first composition is greater than or equal to 30%, and
further wherein the at least one first and at least one second compositions can be packaged separately or together.

102. A makeup kit comprising a cosmetic product, said cosmetic product comprising at least one first composition and at least one second composition, wherein
the at least one first composition comprises a physiologically acceptable medium and, optionally, at least one acrylic polymer, and
the at least one second composition comprises at least one first silicone polymer chosen from polydimethylsiloxanes with a viscosity at 25° C., measured according to standard ASTM D-445, ranging from 5 cSt to 1 0,000.cSt, and at least one second silicone polymer chosen from polydimethylsiloxanes with a viscosity at 25° C., measured according to standard ASTM D-445, ranging from 5,000 cSt to 300,000 cSt,
wherein the viscosity of the at least one second silicone polymer is greater than the viscosity of the at least one first silicone polymer, and
further wherein the at least one first and at least one second compositions can be packaged separately or together.

103. A makeup kit comprising a cosmetic product, said cosmetic product comprising at least one first composition and at least one second composition, wherein
the at least one first composition comprises a physiologically acceptable medium and, optionally, at least one acrylic polymer, and
the at least one second composition comprises at least one first silicone polymer with a viscosity at 25° C., measured according to standard ASTM D-445, ranging from 50 cSt to 1,000 cSt, and at least one second silicone polymer with a viscosity at. 25° C., measured according to standard ASTM D-445, ranging from 30,000 cSt to 300,000 cSt,
wherein the at least one first and at least one second compositions can be packaged separately or together.

104. A makeup kit comprising a cosmetic product, said cosmetic product comprising at least one first composition and at least one second composition, wherein
the at least one first composition comprises a physiologically acceptable medium and, optionally, at least one acrylic polymer, and
the at least one second composition comprises at least one silicone polymer with a viscosity at 25° C., measured according to standard ASTM D-445, ranging from 5 cSt to 10,000 cSt, the at least one silicone polymer being present in a sufficient amount in the at least one second composition such that the staying power of a deposit of the product resulting from the application of the at least one first and at least one second compositions is greater than 65%, and
wherein the at least one first and at least one second compositions can be packaged separately or together.

105. A process for making up the skin and/or the lips and/or the integuments, comprising applying to the skin, the lips and/or the integuments at least one coat of at least one first composition comprising a physiologically acceptable medium and, optionally, at least one acrylic polymer, and applying, onto all or part of the at least one coat of the at least one first composition, at least one coat of at least one second composition comprising at least one silicone polymer with a viscosity at 25° C., measured according to standard ASTM D-445, ranging from 5 cSt to 10,000 cSt,
wherein the staying power of the deposit resulting from the successive application of the at least one first composition and of the at least one second composition being greater than 65%.

106. A method for forming a deposit with a staying power of greater than 65% comprising applying at least one second composition comprising at least one silicone polymer with a viscosity at 25° C., measured according to standard ASTM D-445, ranging from 5 cSt to 10,000 cSt,
wherein the at least one second composition is applied onto at least one first composition comprising a physiologically acceptable medium and, optionally, at least one acrylic polymer, and wherein the deposit results from the successive application of the at least one first and the at least one second composition.
